(19) United States
(12) Patent Application Publication
(10) Pub. No.: US 2021/0113598 A1
(43) Pub. Date: Apr. 22, 2021

(54) Combination of MIDH1 Inhibitors and DNA Hypomethylating Agents (HMA)

(71) Applicant: Deutsches Krebsforschungszentrum (DKFZ) Stiftung des öffentlichen Rechts, Heidelberg (DE)

(72) Inventors: Stefan KAULFUSS, Berlin (DE); Michael Jeffers, Ridgewood, NJ (US); Anuhar Chaturvedi, Hannover (DE); Michael Heuser, Hannover (DE)

(21) Appl. No.: 16/634,654

(22) PCT Filed: Jul. 25, 2018
(86) PCT No.: PCT/EP2018/070173

§ 371 Date: Jan. 28, 2020

(30) Foreign Application Priority Data
Aug. 1, 2017 (EP) 17184281.8
Oct. 31, 2017 (EP) 17199489.0
Dec. 1, 2017 (EP) 17204964.5

(51) Int. Cl. A61K 31/706; A61K 31/4184; A61P 35/02

(52) U.S. Cl. CPC A61K 31/706; A61P 35/02; A61K 31/4184

(57) ABSTRACT

The present invention relates to combinations of at least two components, component A and component B, component A being an inhibitor of mlDH 1, and component B being a DNA hypomethylating agent. Another aspect of the present invention relates to the use of such combinations as described supra for the preparation of a medicament for the treatment or prophylaxis of a disease.

The present invention relates to combinations of at least two components, component A and component B, component A being an inhibitor of mutated isocitratdehydrogenase 1 (mIDH1), or a stereoisomer, a tautomer, an N-oxide, a hydrate, a solvate, or a pharmaceutically acceptable salt thereof, and component B being a DNA hypomethylating agent (HMA), or a stereoisomer, a tautomer, an N-oxide, a hydrate, a solvate, or a pharmaceutically acceptable salt thereof.

Another aspect of the present invention relates to the use of such combinations as described herein for the preparation of a medicament for the treatment or prophylaxis of a disease, particularly for the treatment of cancer, more particularly for the treatment of IDH1 mutated AML.

Another aspect of the present invention relates to the use of a mIDH1 inhibitor as a sensitizer of cells to HMA.

Yet another aspect of the present invention relates to methods of treatment or prophylaxis of a cancer in a subject, comprising administering to said subject a therapeutically effective amount of a combination as described herein.

Further, the present invention relates to a kit comprising a combination of:
one or more components A, as defined herein, or a stereoisomer, a tautomer, an N-oxide, a hydrate, a solvate, or a pharmaceutically acceptable salt thereof;
a component B, as defined supra, or a stereoisomer, a tautomer, an N-oxide, a hydrate, a solvate, or a pharmaceutically acceptable salt thereof; and optionally
one or more pharmaceutical agents C;

in which optionally either or both of said components A and Bare in the form of a pharmaceutical formulation which is ready for use to be administered simultaneously, concurrently, separately or sequentially.

### BACKGROUND

Mutations in the metabolic enzyme isocitrate dehydrogenase 1 (IDH1) are found in 6% of patients with acute myeloid leukemia (AML) and in several other tumors.

Mutant IDH1 produces R-2-hydroxyglutarate (R-2HG), which induces histone and DNA hypermethylation through inhibition of epigenetic regulators, and leads to a block in differentiation to promote tumorigenesis.

Only about one third of Acute Myeloid Leukemia (AML) patients with mutant IDH1 respond to IDH1 inhibitor monotherapy with a median response duration of 7 months, indicating the strong need for combination treatments. 3-(2-{[4-(trifluoromethoxy)phenyl]amino}-1-[(1R,5R)-3,3,5-trimethylcyclohexyl]-1H-benzimidazol-5-yl)propanoic acid (Compound A1), is a highly effective oral pan-mutant IDH1 inhibitor, which has strong anti-leukemic activity in patient derived xenograft (PDX) models of IDH1 mutant acute myloid leukemia in vivo.

### SUMMARY OF THE INVENTION

Surprisingly, it was observed that combinations of mIDH1 inhibitors, or a stereoisomer, a tautomer, an N-oxide, a hydrate, a solvate, or a pharmaceutically acceptable salt thereof, with a HMA, or a stereoisomer, a tautomer, an N-oxide, a hydrate, a solvate, or a pharmaceutically acceptable salt thereof, showed more than additive (synergistic) antiproliferativeactivity.

Therefore, in accordance with a first aspect, the present invention provides combinations of at least two components, component A and component B, component A being a mIDH1 inhibitor, or a stereoisomer, a tautomer, an N-oxide, a hydrate, a solvate, or a pharmaceutically acceptable salt thereof, and component B being a DNA hypomethylating agent (HMA), or a stereoisomer, a tautomer, an N-oxide, a hydrate, a solvate, or a pharmaceutically acceptable salt thereof.

The combinations comprising at least two components, component A and component B, as described and defined herein, are also referred to as “combinations of the present invention”.

### DETAILED DESCRIPTION OF THE INVENTION

### A. Definitions

The term “comprising” when used in the specification includes “consisting of”.

For the purpose of the present invention administration of component A and component B starting simultaneously (e.g. concomitantly) or on the same day is referred to as administration “in parallel”.

For the purpose of the present invention the administration of component A and component B in which each component starts on different days is referred to as sequential.

If it is referred to “above or “supra”, alone or in expressions such as “as mentioned above”, “mentioned above”, or “as defined supra”, within the description it is referred to any of the disclosures made within the specification in any of the preceding pages.

If it is referred to “herein”, alone or in expressions such as “as mentioned herein”, “mentioned herein”, or “as described herein” within the description it is referred to any of the disclosures made within the specification in any of the preceding or subsequent pages.

“suitable” within the sense of the invention means chemically possible to be made by methods within the knowledge of a skilled person.

The terms as mentioned in the present text have preferably the following meanings:

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound (i.e. component A, B or C (when present)) used in the combination of the present invention. An isotopic variation of a compound is defined as one in which at least one atom is replaced by an atom having the same atomic number but an atomic mass different from the atomic mass usually or predominantly found in nature. Examples of isotopes that can be incorporated into a compound used in the combination of the present invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound used in the combination of the present invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence is preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds used in the combination of this invention optionally contain one or more asymmetric centre, depending upon the location and nature of the various substituents desired. Asymmetric carbon atoms is present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds used in the combination of the present invention optionally contain sulphur atoms which are asymmetric, such as an asymmetric sulfoxide, of structure:

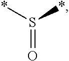

for example,

in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds used in the combination are those which produce the more desirable biological activity. Separated, pure or partially purified isomers and stereoisomers or racemic or diastereomeric mixtures of the compounds used in the combination of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds used in the combination of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound used in the combination of the present invention is achieved by any suitable state of the art method, such as chromatography, especially chiral chromatography, for example.

Further, the compounds used in the combination of the present invention may exist as tautomers.

The present invention includes all possible tautomers of the compounds used in the combination of the present invention as single tautomers, or as any mixture of said tautomers, in any ratio.

Further, the compounds used in the combination of the present invention can exist as N-oxides, which are defined in that at least one nitrogen of the compounds of the present invention is oxidised. The present invention includes all such possible N-oxides.

The present invention also relates to useful forms of the compounds used in the combination as disclosed herein, such as metabolites, hydrates, solvates, prodrugs, salts, in particular pharmaceutically acceptable salts, and co-precipitates.

The compounds used in the combination of the present invention can exist as a hydrate, or as a solvate, wherein the compounds contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds used in the combination of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds used in the combination of the present invention may be, for example, an acid-addition salt of a compound bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound used in the combination of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the compounds may be prepared by reaction of the compounds with the appropriate inorganic or organic acid via any of a number of known methods. Alternatively, alkali and alkaline earth metal salts of acidic compounds used in the combination of the invention are prepared by reacting the compounds with the appropriate base via a variety of known methods.

The present invention includes all possible salts of the compounds used in the combination of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

As used herein, the term “in vivo hydrolysable ester” is understood as meaning an in vivo hydrolysable ester of a compound used in the combination of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl and C₁-C₆-alkoxycarbonyloxyethyl esters, e.g. 1-methoxycarbonyloxyethyl, and may be formed at any carboxy group in the compounds used in the combination of this invention.

An in vivo hydrolysable ester of a compound used in the combination of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds used in the combination of the present invention, either as single polymorph, or as a mixture of more than one polymorph, in any ratio.

In the context of the properties of the compounds used in the combination of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein component A and component Bare present together in one unit dosage or in a single entity. One example of a “fixed combination” is a pharmaceutical composition wherein the said component A and the said component B are present in admixture for simultaneous administration, such as in a formulation. Another example of a “fixed combination” is a pharmaceutical combination wherein the said component A and the said component B are present in one unit without being in admixture.

A non-fixed combination or “kit-of-parts” in the present invention is used as known to persons skilled in the art and is defined as a combination wherein the said component A and the said component B (and optionally component C) are present in more than one unit. One example of a non-fixed combination or kit-of-parts is a combination wherein the said component A and the said component B (and optionally component C) are present separately, for example in different and separate pharmaceutical compositions.

The components of the non-fixed combination or kit-of-parts may be administered separately, sequentially, simultaneously, concurrently or chronologically staggered.

Any such combination is covered by the present invention.

It is further to be understood that embodiments disclosed herein are not meant to be understood as individual embodiments which would not relate to one another. Features discussed with one embodiment or aspect of the invention are meant to be disclosed also in connection with other embodiments or aspects of the invention shown herein. If, in one case, a specific feature is not disclosed with one embodiment or aspect of the invention, but with another, the skilled person would understand that does not necessarily mean that said feature is not meant to be disclosed with said other embodiment or aspect of the invention. The skilled person would understand that it is the gist of this application to disclose said feature also for the other embodiment or aspect of the invention, but that just for purposes of clarity and to keep the length of this specification manageable. It is further to be understood that the content of the prior art documents referred to herein is incorporated by reference, e.g., for enablement purposes, namely when e.g. a method is discussed details of which are described in said prior art document. This approach serves to keep the length of this specification manageable.

Component A of the Combination

Component A can be selected from inhibitors of mIDH1, or a stereoisomer, a tautomer, an N-oxide, a hydrate, a solvate, or a pharmaceutically acceptable salt thereof specifically or generically disclosed herein and/or in the publications mentioned herein, which are incorporated herein by reference.

In an embodiment Component A is selected from the group of mIDH1 inhibitors generically or specifically disclosed in

WO2015/121210,

WO2015/121209,

WO2016/062677,

WO2016/062770,

WO2016/198322,

WO2017/005674,

WO2017/009325,

WO2017/012967,

WO2017/016992, or

WO2017/017046,

which are incorporated by reference herein.

In accordance with a further embodiment, component A is a compound selected from the group consisting of:
tert-butyl{[1-(3,3,5,5-tetramethylcyclohexyl)-2-{[4-(trifluoromethoxy)phenyl]amino}-1H-benzimidazol-5-yl]oxy}acetate,
{[1-(3,3,5,5-tetramethylcyclohexyl)-2-{[4-(trifluoromethoxy)phenyl]amino}-1H-benzimidazol-5-yl]oxy}acetic acid,
(±) methyl (2E)-3-(2-{[4-(trifluoromethoxy)phenyl]amino}-1-[(cis)-3,3,5-trimethylcyclohexyl]-1H-benzimidazol-5-yl)acrylate,
methyl (2E)-3-(2-{[4-(trifluoromethoxy)phenyl]amino}-1-[(1S,5S)-3,3,5-trimethylcyclohexyl]-1H-benzimidazol-5-yl)acrylate,
methyl (2E)-3-(2-{[4-(trifluoromethoxy)phenyl]amino}-1-[(1R,5R)-3,3,5-trimethylcyclohexyl]-1H-benzimidazol-5-yl)acrylate,
(±) (2E)-3-(2-{[4-(trifluoromethoxy)phenyl]amino}-1-[(cis)-3,3,5-trimethylcyclohexyl]-1H-benzimidazol-5-yl)acrylic acid,
(2E)-3-(2-{[4-(trifluoromethoxy)phenyl]amino}-1-[(1S,5S)-3,3,5-trimethylcyclohexyl]-1H-benzimidazol-5-yl)acrylic acid,
(2E)-3-(2-{[4-(trifluoromethoxy)phenyl]amino}-1-[(1R,5R)-3,3,5-trimethylcyclohexyl]-1H-benzimidazol-5-yl)acrylic acid,
(±) methyl (2E)-3-{2-({4-[(trifluoromethyl)sulfanyl]phenyl}amino)-1-[(cis)-3,3,5-trimethylcyclohexyl]-1H-benzimidazol-5-yl}acrylate,
methyl (2E)-3-{2-({4-[(trifluoromethyl)sulfanyl]phenyl}amino)-1-[(1S,5S)-3,3,5-trimethylcyclohexyl]-1H-benzimidazol-5-yl}acrylate,
methyl (2E)-3-{2-({4-[(trifluoromethyl)sulfanyl]phenyl}amino)-1-[(1R,5R)-3,3,5-trimethylcyclohexyl]-1H-benzimidazol-5-yl}acrylate,
(±) methyl 3-(2-{[4-(trifluoromethoxy)phenyl]amino}-1-[(cis)-3,3,5-trimethylcyclohexyl]-1H-benzimidazol-5-yl)propanoate,
methyl 3-(2-{[4-(trifluoromethoxy)phenyl]amino}-1-[(1S,5S)-3,3,5-trimethyl-cyclohexyl]-1H-benzimidazol-5-yl)propanoate,
methyl 3-(2-{[4-(trifluoromethoxy)phenyl]amino}-1-[(1R,5R)-3,3,5-trimethyl-cyclohexyl]-1H-benzimidazol-5-yl)propanoate,
(±) methyl 3-(2-{[4-(trifluoromethyl)phenyl]amino}-1-[(cis)-3,3,5-trimethylcyclohexyl]-1H-benzimidazol-5-yl)propanoate,
methyl 3-(2-{[4-(trifluoromethyl)phenyl]amino}-1-[(1S,5S)-3,3,5-trimethylcyclohexyl]-1H-benzimidazol-5-yl)propanoate,
methyl 3-(2-{[4-(trifluoromethyl)phenyl]amino}-1-[(1R,5R)-3,3,5-trimethylcyclohexyl]-1H-benzimidazol-5-yl)propanoate,
(±) (2E)-3-(2-{[4-(trifluoromethoxy)phenyl]amino}-1-[(cis)-3,3,5-trimethylcyclohexyl]-1H-benzimidazol-5-yl)acrylamide,
(2E)-3-(2-{[4-(trifluoromethoxy)phenyl]amino}-1-[(1S,5S)-3,3,5-trimethylcyclohexyl]-1H-benzimidazol-5-yl)acrylamide,
(2E)-3-(2-{[4-(trifluoromethoxy)phenyl]amino}-1-[(1R,5R)-3,3,5-trimethylcyclohexyl]-1H-benzimidazol-5-yl)acrylamide,
(±) 3-(2-{[4-(trifluoromethoxy)phenyl]amino}-1-[(cis)-3,3,5-trimethylcyclohexyl]-1H-benzimidazol-5-yl)propanamide,
3-(2-{[4-(trifluoromethoxy)phenyl]amino}-1-[(1S,5S)-3,3,5-trimethylcyclohexyl]-1H-benzimidazol-5-yl)propanamide,
3-(2-{[4-(trifluoromethoxy)phenyl]amino}-1-[(1R,5R)-3,3,5 trimethylcyclohexyl]-1H-benzimidazol-5-yl)propanamide,
(±) 3-(2-{[4-(trifluoromethoxy)phenyl]amino}-1-[(cis)-3,3,5-trimethylcyclohexyl]-1H-benzimidazol-5-yl)propanoic acid,
3-(2-{[4-(trifluoromethoxy)phenyl]amino}-1-[(1S,5S)-3,3,5-trimethylcyclohexyl]-1H-benzimidazol-5-yl)propanoic acid,
3-(2-{[4-(trifluoromethoxy)phenyl]amino}-1-[(1R,5R)-3,3,5-trimethylcyclohexyl]-1H-benzimidazol-5-yl)propanoic acid,
(±) 3-(2-{[4-(trifluoromethyl)phenyl]amino}-1-[(cis)-3,3,5-trimethylcyclohexyl]-1H-benzimidazol-5-yl)propanoic acid,
3-(2-{[4-(trifluoromethyl)phenyl]amino}-1-[(1S,5S)-3,3,5-trimethylcyclohexyl]-1H-benzimidazol-5-yl)propanoic acid,
3-(2-{[4-(trifluoromethyl)phenyl]amino}-1-[(1R,5R)-3,3,5-trimethylcyclohexyl]-1H-benzimidazol-5-yl)propanoic acid,
(±) (2E)-N,N-dimethyl-3-(2-{[4-(trifluoromethoxy)phenyl]amino}-1-[(cis)-3,3,5-trimethylcyclohexyl]-1H-benzimidazol-5-yl)acrylamide,
(2E)-N,N-dimethyl-3-(2-{[4-(trifluoromethoxy)phenyl]amino}-1-[(1S,5S)-3,3,5-trimethylcyclohexyl]-1H-benzimidazol-5-yl)acrylamide,
(2E)-N,N-dimethyl-3-(2-{[4-(trifluoromethoxy)phenyl]amino}-1-[(1R,5R)-3,3,5-trimethylcyclohexyl]-1H-benzimidazol-5-yl)acrylamide,
(±) N,N-dimethyl-3-(2-{[4-(trifluoromethoxy)phenyl]amino}-1-[(cis)-3,3,5-trimethylcyclohexyl]-1H-benzimidazol-5-yl)propanamide,
N,N-dimethyl-3-(2-{[4-(trifluoromethoxy)phenyl]amino}-1-[(1S,5S)-3,3,5-trimethylcyclohexyl]-1H-benzimidazol-5-yl)propanamide,
N,N-dimethyl-3-(2-{[4-(trifluoromethoxy)phenyl]amino}-1-[(1R,5R)-3,3,5-trimethylcyclohexyl]-1H-benzimidazol-5-yl)propanamide,
(±) ({2-[(4-ethoxyphenyl)amino]-1-[(cis)-3,3,5-trimethylcyclohexyl]-1H-benzimidazol-5-yl}oxy)acetic acid,
({2-[(4-ethoxyphenyl)amino]-1-[(1S,5S)-3,3,5-trimethylcyclohexyl]-1H-benzimidazol-5-yl}oxy)acetic acid,
({2-[(4-ethoxyphenyl)amino]-1-[(1R,5R)-3,3,5-trimethylcyclohexyl]-1H-benzimidazol-5-yl}oxy)acetic acid,
(±) [(2-{[4-(trifluoromethoxy)phenyl]amino}-1-[(cis)-3,3,5-trimethylcyclohexyl]-1H-benzimidazol-5-yl)oxy]acetic acid,
[(2-{[4-(trifluoromethoxy)phenyl]amino}-1-[(1S,5S)-3,3,5-trimethylcyclohexyl]-1H-benzimidazol-5-yl)oxy]acetic acid,
[(2-{[4-(trifluoromethoxy)phenyl]amino}-1-[(1R,5R)-3,3,5-trimethylcyclohexyl]-1H-benzimidazol-5-yl)oxy]acetic acid,
(±) ({2-[(4-isopropoxyphenyl)amino]-1-[(cis)-3,3,5-trimethylcyclohexyl]-1H-benzimidazol-5-yl}oxy)acetic acid,
({2-[(4-isopropoxyphenyl)amino]-1-[(1S,5S)-3,3,5-trimethylcyclohexyl]-1H-benzimidazol-5-yl}oxy)acetic acid,
({2-[(4-isopropoxyphenyl)amino]-1-[(1R,5R)-3,3,5-trimethylcyclohexyl]-1H-benzimidazol-5-yl}oxy)acetic acid,
(±) ({2-[(4-cyanophenyl)amino]-1-[(cis)-3,3,5-trimethylcyclohexyl]-1H-benzimidazol-5-yl}oxy)acetic acid,
({2-[(4-cyanophenyl)amino]-1-[(1 S,5S)-3,3,5-trimethylcyclohexyl]-1H-benzimidazol-5-yl}oxy)acetic acid,
({2-[(4-cyanophenyl)amino]-1-[(1R,5R)-3,3,5-trimethylcyclohexyl]-1H-benzimidazol-5-yl}oxy)acetic acid,
(±) methyl N-{[(2-{[4-(trifluoromethoxy)phenyl]amino}-1-[(cis)-3,3,5-trimethylcyclohexyl]-1H-benzimidazol-5-yl)oxy]acetyl}glycinate,
methyl N-{[(2-{[4-(trifluoromethoxy)phenyl]amino}-1-[(1S,5S)-3,3,5-trimethylcyclohexyl]-1H-benzimidazol-5-yl)oxy]acetyl}glycinate,
methyl N-{[(2-{[4-(trifluoromethoxy)phenyl]amino}-1-[(1R,5R)-3,3,5-trimethylcyclohexyl]-1H-benzimidazol-5-yl)oxy]acetyl}glycinate,
(±) N-cyclopropyl-2-[(2-{[4-(trifluoromethoxy)phenyl]amino}-1-[(cis)-3,3,5-trimethylcyclohexyl]-1H-benzimidazol-5-yl)oxy]acetamide,
N-cyclopropyl-2-[(2-{[4-(trifluoromethoxy)phenyl]amino}-1-[(1S,5S)-3,3,5-trimethylcyclohexyl]-1H-benzimidazol-5-yl)oxy]acetamide,
N-cyclopropyl-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-{[(2-{[4-(trifluoromethoxy)phenyl]amino}-1-[(cis)-3,3,5-trimethylcyclohexyl]-1H-benzimidazol-5-yl)oxy]acetyl}glycine,
N-{[(2-{[4-(trifluoromethoxy)phenyl]amino}-1-[(1S,5S)-3,3,5-trimethylcyclohexyl]-1H-benzimidazol-5-yl)oxy]acetyl}glycine,
N-{[(2-{[4-(trifluoromethoxy)phenyl]amino}-1-[(1R,5R)-3,3,5-trimethylcyclohexyl]-1H-benzimidazol-5-yl)oxy]acetyl}glycine,
(±) methyl N-methyl-N-{[(2-{[4-(trifluoromethoxy)phenyl]amino}-1-[(cis)-3,3,5-trimethylcyclohexyl]-1H-benzimidazol-5-yl)oxy]acetyl}glycinate,
methyl N-methyl-N-{[(2-{[4-(trifluoromethoxy)phenyl]amino}-1-[(1S,5S)-3,3,5-trimethylcyclohexyl]-1H-benzimidazol-5-yl)oxy]acetyl}glycinate,
methyl N-methyl-N-{[(2-{[4-(trifluoromethoxy)phenyl]amino}-1-[(1R,5R)-3,3,5-trimethylcyclohexyl]-1H-benzimidazol-5-yl)oxy]acetyl}glycinate,
(±) N-methyl-N-{[(2-{[4-(trifluoromethoxy)phenyl]amino}-1-[(cis)-3,3,5-trimethylcyclohexyl]-1H-benzimidazol-5-yl)oxy]acetyl}glycine,
N-methyl-N-{[(2-{[4-(trifluoromethoxy)phenyl]amino}-1-[(1S,5S)-3,3,5-trimethylcyclohexyl]-1H-benzimidazol-5-yl)oxy]acetyl}glycine,
N-methyl-N-{[(2-{[4-(trifluoromethoxy)phenyl]amino}-1-[(1R,5R)-3,3,5-trimethylcyclohexyl]-1H-benzimidazol-5-yl)oxy]acetyl}glycine,
(±) 4-[(2-{[4-(trifluoromethoxy)phenyl]amino}-1-[(cis)-3,3,5-trimethylcyclohexyl]-1H-benzimidazol-5-yl)oxy]butanoic acid,
4-[(2-{[4-(trifluoromethoxy)phenyl]amino}-1-[(1S,5S)-3,3,5-trimethylcyclohexyl]-1H-benzimidazol-5-yl)oxy]butanoic acid,
4-[(2-{[4-(trifluoromethoxy)phenyl]amino}-1-[(1R,5R)-3,3,5-trimethylcyclohexyl]-1H-benzimidazol-5-yl)oxy]butanoic acid,
(±) 4-[(2-{[4-(isopropyl)phenyl]amino}-1-[(cis)-3,3,5-trimethylcyclohexyl]-1H-benzimidazol-5-yl)oxy]butanoic acid,
4-[(2-{[4-(isopropyl)phenyl]amino}-1-[(1S,5S)-3,3,5-trimethylcyclohexyl]-1H-benzimidazol-5-yl)oxy]butanoic acid,
4-[(2-{[4-(isopropyl)phenyl]amino}-1-[(1R,5R)-3,3,5-trimethylcyclohexyl]-1H-benzimidazol-5-yl)oxy]butanoic acid,
(±) 4-[(2-{[4-(isopropoxy)phenyl]amino}-1-[(cis)-3,3,5-trimethylcyclohexyl]-1H-benzimidazol-5-yl)oxy]butanoic acid,
4-[(2-{[4-(isopropoxy)phenyl]amino}-1-[(1S,5S)-3,3,5-trimethylcyclohexyl]-1H-benzimidazol-5-yl)oxy]butanoic acid,
4-[(2-{[4-(isopropoxy)phenyl]amino}-1-[(1R,5R)-3,3,5-trimethylcyclohexyl]-1H-benzimidazol-5-yl)oxy]butanoic acid,
(±) 4-[(2-{[4-(trifluoromethyl)phenyl]amino}-1-[(cis)-3,3,5-trimethylcyclohexyl]-1H-benzimidazol-5-yl)oxy]butanoic acid,
4-[(2-{[4-(trifluoromethyl)phenyl]amino}-1-[(1S,5S)-3,3,5-trimethylcyclohexyl]-1H-benzimidazol-5-yl)oxy]butanoic acid,
4-[(2-{[4-(trifluoromethyl)phenyl]amino}-1-[(1R,5R)-3,3,5-trimethylcyclohexyl]-1H-benzimidazol-5-yl)oxy]butanoic acid,
(±) methyl (2E)-3-(2-{[4-(trifluoromethyl)phenyl]amino}-1-[(cis)-3,3,5-trimethylcyclohexyl]-1H-benzimidazol-5-yl)acrylate,
methyl (2E)-3-(2-{[4-(trifluoromethyl)phenyl]amino}-1-[(1S,5S)-3,3,5-trimethylcyclohexyl]-1H-benzimidazol-5-yl)acrylate,
methyl (2E)-3-(2-{[4-(trifluoromethyl)phenyl]amino}-1-[(1R,5R)-3,3,5-trimethylcyclohexyl]-1H-benzimidazol-5-yl)acrylate,
(±) methyl 3-(6-methyl-2-{[4-(trifluoromethoxy)phenyl]amino}-1-[(cis)-3,3,5-trimethylcyclohexyl]-1H-benzimidazol-5-yl)propanoate,
methyl 3-(6-methyl-2-{[4-(trifluoromethoxy)phenyl]amino}-1-[(1S,5S)-3,3,5-trimethylcyclohexyl]-1H-benzimidazol-5-yl)propanoate,
methyl 3-(6-methyl-2-{[4-(trifluoromethoxy)phenyl]amino}-1-[(1R,5R)-3,3,5-trimethylcyclohexyl]-1H-benzimidazol-5-yl)propanoate,
(±) methyl 3-(6-methyl-2-{[4-(propan-2-yloxy)phenyl]amino}-1-[(cis)-3,3,5-trimethylcyclohexyl]-1H-benzimidazol-5-yl)propanoate,
methyl 3-(6-methyl-2-{[4-(propan-2-yloxy)phenyl]amino}-1-[(1S,5S)-3,3,5-trimethylcyclohexyl]-1H-benzimidazol-5-yl)propanoate,
methyl 3-(6-methyl-2-{[4-(propan-2-yloxy)phenyl]amino}-1-[(1R,5R)-3,3,5-trimethylcyclohexyl]-1H-benzimidazol-5-yl)propanoate,
(±) methyl 3-(6-methyl-2-{[4-(propan-2-yl)phenyl]amino}-1-[(cis)-3,3,5-trimethylcyclohexyl]-1H-benzimidazol-5-yl)propanoate,
methyl 3-(6-methyl-2-{[4-(propan-2-yl)phenyl]amino}-1-[(1S,5S)-3,3,5-trimethylcyclohexyl]-1H-benzimidazol-5-yl)propanoate,
methyl 3-(6-methyl-2-{[4-(propan-2-yl)phenyl]amino}-1-[(1R,5R)-3,3,5-trimethylcyclohexyl]-1H-benzimidazol-5-yl)propanoate,
(±) methyl 3-(6-methyl-2-{[4-(trifluoromethyl)phenyl]amino}-1-[(cis)-3,3,5-trimethylcyclohexyl]-1H-benzimidazol-5-yl)propanoate,
methyl 3-(6-methyl-2-{[4-(trifluoromethyl)phenyl]amino}-1-[(1S,5S)-3,3,5-trimethylcyclohexyl]-1H-benzimidazol-5-yl)propanoate,
methyl 3-(6-methyl-2-{[4-(trifluoromethyl)phenyl]amino}-1-[(1R,5R)-3,3,5-trimethylcyclohexyl]-1H-benzimidazol-5-yl)propanoate,
(±) 3-(6-methyl-2-{[4-(trifluoromethoxy)phenyl]amino}-1-[(cis)-3,3,5-trimethylcyclohexyl]-1H-benzimidazol-5-yl)propanoic acid,
3-(6-methyl-2-{[4-(trifluoromethoxy)phenyl]amino}-1-[(1S,5S)-3,3,5-trimethylcyclohexyl]-1H-benzimidazol-5-yl)propanoic acid,
3-(6-methyl-2-{[4-(trifluoromethoxy)phenyl]amino}-1-[(1R,5R)-3,3,5-trimethylcyclohexyl]-1H-benzimidazol-5-yl)propanoic acid,
(±) 3-(6-methyl-2-{[4-(propan-2-yloxy)phenyl]amino}-1-[(cis)-3,3,5-trimethylcyclohexyl]-1H-benzimidazol-5-yl)propanoic acid,
3-(6-methyl-2-{[4-(propan-2-yloxy)phenyl]amino}-1-[(1R,5R)-3,3,5-trimethylcyclohexyl]-1H-benzimidazol-5-yl)propanoic acid,
3-(6-methyl-2-{[4-(propan-2-yloxy)phenyl]amino}-1-[(1S,5S)-3,3,5-trimethylcyclohexyl]-1H-benzimidazol-5-yl)propanoic acid,
(±) 3-(6-methyl-2-{[4-(propan-2-yl)phenyl]amino}-1-[(cis)-3,3,5-trimethylcyclohexyl]-1H-benzimidazol-5-yl)propanoic acid,
3-(6-methyl-2-{[4-(propan-2-yl)phenyl]amino}-1-[(1S,5S)-3,3,5-trimethylcyclohexyl]-1H-benzimidazol-5-yl)propanoic acid,
3-(6-methyl-2-{[4-(propan-2-yl)phenyl]amino}-1-[(1R,5R)-3,3,5-trimethylcyclohexyl]-1H-benzimidazol-5-yl)propanoic acid,
(±) 3-(6-methyl-2-{[4-(trifluoromethyl)phenyl]amino}-1-[(cis)-3,3,5-trimethylcyclohexyl]-1H-benzimidazol-5-yl)propanoic acid,
3-(6-methyl-2-{[4-(trifluoromethyl)phenyl]amino}-1-[(1S,5S)-3,3,5-trimethylcyclohexyl]-1H-benzimidazol-5-yl)propanoic acid,
3-(6-methyl-2-{[4-(trifluoromethyl)phenyl]amino}-1-[(1R,5R)-3,3,5-trimethylcyclohexyl]-1H-benzimidazol-5-yl)propanoic acid,
(±) methyl 3-(6-methoxy-2-{[4-(trifluoromethoxy)phenyl]amino}-1-[(cis)-3,3,5-trimethylcyclohexyl]-1H-benzimidazol-5-yl)propanoate,
methyl 3-(6-methoxy-2-{[4-(trifluoromethoxy)phenyl]amino}-1-[(1S,5S)-3,3,5-trimethylcyclohexyl]-1H-benzimidazol-5-yl)propanoate,
methyl 3-(6-methoxy-2-{[4-(trifluoromethoxy)phenyl]amino}-1-[(1R,5R)-3,3,5-trimethylcyclohexyl]-1H-benzimidazol-5-yl)propanoate,
(±) methyl 3-(6-methoxy-2-{[4-(propan-2-yl)phenyl]amino}-1-[(cis)-3,3,5-trimethylcyclohexyl]-1H-benzimidazol-5-yl)propanoate,
methyl 3-(6-methoxy-2-{[4-(propan-2-yl)phenyl]amino}-1-[(1S,5S)-3,3,5-trimethylcyclohexyl]-1H-benzimidazol-5-yl)propanoate,
methyl 3-(6-methoxy-2-{[4-(propan-2-yl)phenyl]amino}-1-[(1R,5R)-3,3,5-trimethylcyclohexyl]-1H-benzimidazol-5-yl)propanoate,
(±) methyl 3-(6-methoxy-2-{[4-(trifluoromethyl)phenyl]amino}-1-[(cis)-3,3,5-trimethylcyclohexyl]-1H-benzimidazol-5-yl)propanoate,
methyl 3-(6-methoxy-2-{[4-(trifluoromethyl)phenyl]amino}-1-[(1S,5S)-3,3,5-trimethylcyclohexyl]-1H-benzimidazol-5-yl)propanoate,
methyl 3-(6-methoxy-2-{[4-(trifluoromethyl)phenyl]amino}-1-[(1R,5R)-3,3,5-trimethylcyclohexyl]-1H-benzimidazol-5-yl)propanoate,
(±) 3-(6-methoxy-2-{[4-(trifluoromethoxy)phenyl]amino}-1-[(cis)-3,3,5-trimethylcyclohexyl]-1H-benzimidazol-5-yl)propanoic acid,
3-(6-methoxy-2-{[4-(trifluoromethoxy)phenyl]amino}-1-[(1S,5S)-3,3,5-trimethylcyclohexyl]-1H-benzimidazol-5-yl)propanoic acid,
3-(6-methoxy-2-{[4-(trifluoromethoxy)phenyl]amino}-1-[(1R,5R)-3,3,5-trimethylcyclohexyl]-1H-benzimidazol-5-yl)propanoic acid,
(±) 3-(6-methoxy-2-{[4-(propan-2-yl)phenyl]amino}-1-[(cis)-3,3,5-trimethylcyclohexyl]-1H-benzimidazol-5-yl)propanoic acid,
3-(6-methoxy-2-{[4-(propan-2-yl)phenyl]amino}-1-[(1S,5S)-3,3,5-trimethylcyclohexyl]-1H-benzimidazol-5-yl)propanoic acid,
3-(6-methoxy-2-{[4-(propan-2-yl)phenyl]amino}-1-[(1R,5R)-3,3,5-trimethylcyclohexyl]-1H-benzimidazol-5-yl)propanoic acid,
(±) 3-(6-methoxy-2-{[4-(trifluoromethyl)phenyl]amino}-1-[(cis)-3,3,5-trimethylcyclohexyl]-1H-benzimidazol-5-yl)propanoic acid,
3-(6-methoxy-2-{[4-(trifluoromethyl)phenyl]amino}-1-[(1S,5S)-3,3,5-trimethylcyclohexyl]-1H-benzimidazol-5-yl)propanoic acid,
3-(6-methoxy-2-{[4-(trifluoromethyl)phenyl]amino}-1-[(1R,5R)-3,3,5-trimethylcyclohexyl]-1H-benzimidazol-5-yl)propanoic acid,
(±) methyl 3-(2-{[4-(propan-2-yloxy)phenyl]amino}-1-[(cis)-3,3,5-trimethylcyclohexyl]-1H-benzimidazol-5-yl)propanoate,
methyl 3-(2-{[4-(propan-2-yloxy)phenyl]amino}-1-[(1S,5S)-3,3,5-trimethylcyclohexyl]-1H-benzimidazol-5-yl)propanoate,
methyl 3-(2-{[4-(propan-2-yloxy)phenyl]amino}-1-[(1R,5R)-3,3,5-trimethylcyclohexyl]-1H-benzimidazol-5-yl)propanoate,
(±) methyl 3-(2-{[4-(propan-2-yl)phenyl]amino}-1-[(cis)-3,3,5-trimethylcyclohexyl]-1H-benzimidazol-5-yl)propanoate,
methyl 3-(2-{[4-(propan-2-yl)phenyl]amino}-1-[(1R,5R)-3,3,5-trimethylcyclohexyl]-1H-benzimidazol-5-yl)propanoate,
methyl 3-(2-{[4-(propan-2-yl)phenyl]amino}-1-[(1S,5S)-3,3,5-trimethylcyclohexyl]-1H-benzimidazol-5-yl)propanoate,
(±) 3-(2-{[4-(propan-2-yloxy)phenyl]amino}-1-[(cis)-3,3,5-trimethylcyclohexyl]-1H-benzimidazol-5-yl)propanoic acid,
3-(2-{[4-(propan-2-yloxy)phenyl]amino}-1-[(1S,5S)-3,3,5-trimethylcyclohexyl]-1H-benzimidazol-5-yl)propanoic acid,
3-(2-{[4-(propan-2-yloxy)phenyl]amino}-1-[(1R,5R)-3,3,5-trimethylcyclohexyl]-1H-benzimidazol-5-yl)propanoic acid,
(±) 3-(2-{[4-(propan-2-yl)phenyl]amino}-1-[(cis)-3,3,5-trimethylcyclohexyl]-1H-benzimidazol-5-yl)propanoic acid,
3-(2-{[4-(propan-2-yl)phenyl]amino}-1-[(1S,5S)-3,3,5-trimethylcyclohexyl]-1H-benzimidazol-5-yl)propanoic acid,
3-(2-{[4-(propan-2-yl)phenyl]amino}-1-[(1R,5R)-3,3,5-trimethylcyclohexyl]-1H-benzimidazol-5-yl)propanoic acid,
(±) methyl 3-{2-[(4-isopropoxyphenyl)amino]-6-methoxy-1-[(cis)-3,3,5-trimethylcyclohexyl]-1H-benzimidazol-5-yl}propanoate,
methyl 3-{2-[(4-isopropoxyphenyl)amino]-6-methoxy-1-[(1S,5S)-3,3,5-trimethylcyclohexyl]-1H-benzimidazol-5-yl}propanoate,
methyl 3-{2-[(4-isopropoxyphenyl)amino]-6-methoxy-1-[(1R,5R)-3,3,5-trimethylcyclohexyl]-1H-benzimidazol-5-yl}propanoate,
(±) 3-{2-[(4-isopropoxyphenyl)amino]-6-methoxy-1-[(cis)-3,3,5-trimethylcyclohexyl]-1H-benzimidazol-5-yl}propanoic acid,
3-{2-[(4-isopropoxyphenyl)amino]-6-methoxy-1-[(1S,5S)-3,3,5-trimethylcyclohexyl]-1H-benzimidazol-5-yl}propanoic acid,
3-{2-[(4-isopropoxyphenyl)amino]-6-methoxy-1-[(1R,5R)-3,3,5-trimethylcyclohexyl]-1H-benzimidazol-5-yl}propanoic acid,
(±) 4-({5-(2-carboxyethyl)-6-methoxy-1-[(cis)-3,3,5-trimethylcyclohexyl]-1H-benzimidazol-2-yl}amino)benzoic acid,
4-({5-(2-carboxyethyl)-6-methoxy-1-[(1S,5S)-3,3,5-trimethylcyclohexyl]-1H-benzimidazol-2-yl}amino)benzoic acid,
4-({5-(2-carboxyethyl)-6-methoxy-1-[(1R,5R)-3,3,5-trimethylcyclohexyl]-1H-benzimidazol-2-yl}amino)benzoic acid,
(±) 2-[(2-{[4-(trifluoromethoxy)phenyl]amino}-1-[(cis)-3,3,5-trimethylcyclohexyl]-1H-benzimidazol-5-yl)oxy]-N-{[3-(trifluoromethyl)pyridin-2-yl]methyl}acetamide,
2-[(2-{[4-(trifluoromethoxy)phenyl]amino}-1-[(1S,5S)-3,3,5-trimethylcyclohexyl]-1H-benzimidazol-5-yl)oxy]-N-{[3-(trifluoromethyl)pyridin-2-yl]methyl}acetamide,
2-[(2-{[4-(trifluoromethoxy)phenyl]amino}-1-[(1R,5R)-3,3,5-trimethylcyclohexyl]-1H-benzimidazol-5-yl)oxy]-N-{[3-(trifluoromethyl)pyridin-2-yl]methyl}acetamide,
(±) N-(2-chlorophenyl)-2-[(2-{[4-(trifluoromethoxy)phenyl]amino}-1-[(cis)-3,3,5-trimethylcyclohexyl]-1H-benzimidazol-5-yl)oxy]acetamide,
N-(2-chlorophenyl)-2-[(2-{[4-(trifluoromethoxy)phenyl]amino}-1-[(1S,5S)-3,3,5-trimethylcyclohexyl]-1H-benzimidazol-5-yl)oxy]acetamide,
N-(2-chlorophenyl)-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-[(3-methylpyridin-2-yl)methyl]-2-[(2-{[4-(trifluoromethoxy)phenyl]amino}-1-[(cis)-3,3,5-trimethylcyclohexyl]-1H-benzimidazol-5-yl)oxy]acetamide,
N-[(3-methylpyridin-2-yl)methyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[(3-methylpyridin-2-yl)methyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-[(3-fluoropyridin-2-yl)methyl]-2-[(2-{[4-(trifluoromethoxy)phenyl]amino}-1-[(cis)-3,3,5-trimethylcyclohexyl]-1H-benzimidazol-5-yl)oxy]acetamide,
N-[(3-fluoropyridin-2-yl)methyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[(3-fluoropyridin-2-yl)methyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-{[3-chloro-5-(trifluoromethyl)pyridin-2-yl]methyl}-2-[(2-{[4-(trifluoromethoxy)phenyl]amino}-1-[(cis)-3,3,5-trimethylcyclohexyl]-1H-benzimidazol-5-yl)oxy]acetamide,
N-{[3-chloro-5-(trifluoromethyl)pyridin-2-yl]methyl}-2-[(2-{[4-(trifluoromethoxy)phenyl]amino}-1-[(1S,5S)-3,3,5-trimethylcyclohexyl]-1H-benzimidazol-5-yl)oxy]acetamide,
N-{[3-chloro-5-(trifluoromethyl)pyridin-2-yl]methyl}-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-{[3-chloro-5-(trifluoromethyl)pyridin-2-yl]methyl}-2-[(2-{[4-(trifluoromethoxy)phenyl]amino}-1-[(cis)-3,3,5-trimethylcyclohexyl]-1H-benzimidazol-5-yl)oxy]acetamide,
N-{[3-chloro-5-(trifluoromethyl)pyridin-2-yl]methyl}-2-[(2-{[4-(trifluoromethoxy)phenyl]amino}-1-[(1S,5S)-3,3,5-trimethylcyclohexyl]-1H-benzimidazol-5-yl)oxy]acetamide,
N-{[3-chloro-5-(trifluoromethyl)pyridin-2-yl]methyl}-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-[3-(trifluoromethoxy)phenyl]-2-[(2-{[4-(trifluoromethoxy)phenyl]amino}-1-[(cis)-3,3,5-trimethylcyclohexyl]-1H-benzimidazol-5-yl)oxy]acetamide,
N-[3-(trifluoromethoxy)phenyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[3-(trifluoromethoxy)phenyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) 2-[(2-{[4-(trifluoromethoxy)phenyl]amino}-1-[(cis)-3,3,5-trimethylcyclohexyl]-1H-benzimidazol-5-yl)oxy]-N-[4-(trifluoromethyl)phenyl]acetamide,
2-[(2-{[4-(trifluoromethoxy)phenyl]amino}-1-[(1S,5S)-3,3,5-trimethylcyclohexyl]-1H-benzimidazol-5-yl)oxy]-N-[4-(trifluoromethyl)phenyl]acetamide,
2-[(2-{[4-(trifluoromethoxy)phenyl]amino}-1-[(1R,5R)-3,3,5-trimethylcyclohexyl]-1H-benzimidazol-5-yl)oxy]-N-[4-(trifluoromethyl)phenyl]acetamide,
(±) 2-[(2-{[4-(trifluoromethoxy)phenyl]amino}-1-[(cis)-3,3,5-trimethylcyclohexyl]-1H-benzimidazol-5-yl)oxy]-N-[3-(trifluoromethyl)phenyl]acetamide,
2-[(2-{[4-(trifluoromethoxy)phenyl]amino}-1-[(1S,5S)-3,3,5-trimethylcyclohexyl]-1H-benzimidazol-5-yl)oxy]-N-[3-(trifluoromethyl)phenyl]acetamide,
2-[(2-{[4-(trifluoromethoxy)phenyl]amino}-1-[(1R,5R)-3,3,5-trimethylcyclohexyl]-1H-benzimidazol-5-yl)oxy]-N-[3-(trifluoromethyl)phenyl]acetamide,
(±) N-[3-(difluoromethoxy)phenyl]-2-[(2-{[4-(trifluoromethoxy)phenyl]amino}-1-[(cis)-3,3,5-trimethylcyclohexyl]-1H-benzimidazol-5-yl)oxy]acetamide,
N-[3-(difluoromethoxy)phenyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[3-(difluoromethoxy)phenyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) methyl-3-({[(2-{[4-(trifluoromethoxy)phenyl]amino}-1-[(cis)-3,3,5-trimethylcyclohexyl]-1H-benzimidazol-5-yl)oxy]acetyl}amino)benzoate,
methyl-3-({[(2-{[4-(trifluoromethoxy)phenyl]amino}-1-[(1S,5S)-3,3,5-trimethylcyclohexyl]-1H-benzimidazol-5-yl)oxy]acetyl}amino)benzoate,
methyl-3-({[(2-{[4-(trifluoromethoxy)phenyl]amino}-1-[(1R,5R)-3,3,5-trimethylcyclohexyl]-1H-benzimidazol-5-yl)oxy]acetyl}amino)benzoate,
(±) N-[2-chloro-5-(difluoromethyl)phenyl]-2-[(2-{[4-(trifluoromethoxy)phenyl]amino}-1-[(cis)-3,3,5-trimethylcyclohexyl]-1H-benzimidazol-5-yl)oxy]acetamide,
N-[2-chloro-5-(difluoromethyl)phenyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[2-chloro-5-(difluoromethyl)phenyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-[4-(difluoromethoxy)phenyl]-2-[(2-{[4-(trifluoromethoxy)phenyl]amino}-1-[(cis)-3,3,5-trimethylcyclohexyl]-1H-benzimidazol-5-yl)oxy]acetamide,
N-[4-(difluoromethoxy)phenyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[4-(difluoromethoxy)phenyl]-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-(2-methylphenyl)-2-[(2-{[4-(trifluoromethoxy)phenyl]amino}-1-[(cis)-3,3,5-trimethylcyclohexyl]-1H-benzimidazol-5-yl)oxy]acetamide,
N-(2-methylphenyl)-2-[(2-{[4-(trifluoromethoxy)phenyl]amino}-1-[(1S,5S)-3,3,5-trimethylcyclohexyl]-1H-benzimidazol-5-yl)oxy]acetamide,
N-(2-methylphenyl)-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-(3-methylphenyl)-2-[(2-{[4-(trifluoromethoxy)phenyl]amino}-1-[(cis)-3,3,5-trimethylcyclohexyl]-1H-benzimidazol-5-yl)oxy]acetamide,
N-(3-methylphenyl)-2-[(2-{[4-(trifluoromethoxy)phenyl]amino}-1-[(1S,5S)-3,3,5-trimethylcyclohexyl]-1H-benzimidazol-5-yl)oxy]acetamide,
N-(3-methylphenyl)-2-[(2-{[4-(trifluoromethoxy)phenyl]amino}-1-[(1R,5R)-3,3,5-trimethylcyclohexyl]-1H-benzimidazol-5-yl)oxy]acetamide,
(±) N-[4-(trifluoromethoxy)phenyl]-2-[(2-{[4-(trifluoromethoxy)phenyl]amino}-1-[(cis)-3,3,5-trimethylcyclohexyl]-1H-benzimidazol-5-yl)oxy]acetamide,
N-[4-(trifluoromethoxy)phenyl]-2-[(2-{[4-(trifluoromethoxy)phenyl]amino}-1-[(1S,5S)-3,3,5-trimethylcyclohexyl]-1H-benzimidazol-5-yl)oxy]acetamide,
N-[4-(trifluoromethoxy)phenyl]-2-[(2-{[4-(trifluoromethoxy)phenyl]amino}-1-[(1R,5R)-3,3,5-trimethylcyclohexyl]-1H-benzimidazol-5-yl)oxy]acetamide,
methyl [1-(3,3,5,5-tetramethylcyclohexyl)-2-{[4-(trifluoromethoxy)phenyl]amino}-1H-benzimidazol-5-yl]acetate,
methyl [2-{[4-(propan-2-yloxy)phenyl]amino}-1-(3,3,5,5-tetramethylcyclohexyl)-1H-benzimidazol-5-yl]acetate,
methyl [2-{[4-(propan-2-yl)phenyl]amino}-1-(3,3,5,5-tetramethylcyclohexyl)-1H-benzimidazol-5-yl]acetate,
(±) methyl (2-{[4-(propan-2-yl)phenyl]amino}-1-[(cis)-3,3,5-trimethylcyclohexyl]-1H-benzimidazol-5-yl)acetate,
methyl (2-{[4-(propan-2-yl)phenyl]amino}-1-[(1S,5S)-3,3,5-trimethylcyclohexyl]-1H-benzimidazol-5-yl)acetate,
methyl (2-{[4-(propan-2-yl)phenyl]amino}-1-[(1R,5R)-3,3,5-trimethylcyclohexyl]-1H-benzimidazol-5-yl)acetate,
(±) methyl (2-{[4-(propan-2-yloxy)phenyl]amino}-1-[(cis)-3,3,5-trimethylcyclohexyl]-1H-benzimidazol-5-yl)acetate,
methyl (2-{[4-(propan-2-yloxy)phenyl]amino}-1-[(1S,5S)-3,3,5-trimethylcyclohexyl]-1H-benzimidazol-5-yl)acetate,
methyl (2-{[4-(propan-2-yloxy)phenyl]amino}-1-[(1R,5R)-3,3,5-trimethylcyclohexyl]-1H-benzimidazol-5-yl)acetate,
(±) methyl (2-{[4-(trifluoromethoxy)phenyl]amino}-1-[(cis)-3,3,5-trimethylcyclohexyl]-1H-benzimidazol-5-yl)acetate,
methyl (2-{[4-(trifluoromethoxy)phenyl]amino}-1-[(1S,5S)-3,3,5-trimethylcyclohexyl]-1H-benzimidazol-5-yl)acetate,
methyl (2-{[4-(trifluoromethoxy)phenyl]amino}-1-[(1R,5R)-3,3,5-trimethylcyclohexyl]-1H-benzimidazol-5-yl)acetate,
methyl 3-[1-(3,3,5,5-tetramethylcyclohexyl)-2-{[4-(trifluoromethoxy)phenyl]amino}-1H-benzimidazol-5-yl]propanoate,
methyl [6-methyl-2-{[4-(propan-2-yloxy)phenyl]amino}-1-(3,3,5,5-tetramethylcyclohexyl)-1H-benzimidazol-5-yl]acetate,
methyl [6-methyl-2-{[4-(propan-2-yl)phenyl]amino}-1-(3,3,5,5-tetramethylcyclohexyl)-1H-benzimidazol-5-yl]acetate,
methyl [6-methyl-1-(3,3,5,5-tetramethylcyclohexyl)-2-{[4-(trifluoromethoxy)phenyl]amino}-1H-benzimidazol-5-yl]acetate,
(±) methyl (6-methyl-2-{[4-(propan-2-yloxy)phenyl]amino}-1-[(cis)-3,3,5-trimethylcyclohexyl]-1H-benzimidazol-5-yl)acetate,
methyl (6-methyl-2-{[4-(propan-2-yloxy)phenyl]amino}-1-[(1S,5S)-3,3,5-trimethylcyclohexyl]-1H-benzimidazol-5-yl)acetate,
methyl (6-methyl-2-{[4-(propan-2-yloxy)phenyl]amino}-1-[(1R,5R)-3,3,5-trimethylcyclohexyl]-1H-benzimidazol-5-yl)acetate,
(±) methyl (6-methyl-2-{[4-(propan-2-yl)phenyl]amino}-1-[(cis)-3,3,5-trimethylcyclohexyl]-1H-benzimidazol-5-yl)acetate,
methyl (6-methyl-2-{[4-(propan-2-yl)phenyl]amino}-1-[(1S,5S)-3,3,5-trimethylcyclohexyl]-1H-benzimidazol-5-yl)acetate,
methyl (6-methyl-2-{[4-(propan-2-yl)phenyl]amino}-1-[(1R,5R)-3,3,5-trimethylcyclohexyl]-1H-benzimidazol-5-yl)acetate,
(±) methyl (6-methyl-2-{[4-(trifluoromethoxy)phenyl]amino}-1-[(cis)-3,3,5-trimethylcyclohexyl]-1H-benzimidazol-5-yl)acetate,
methyl (6-methyl-2-{[4-(trifluoromethoxy)phenyl]amino}-1-[(1S,5S)-3,3,5-trimethylcyclohexyl]-1H-benzimidazol-5-yl)acetate,
methyl (6-methyl-2-{[4-(trifluoromethoxy)phenyl]amino}-1-[(1R,5R)-3,3,5-trimethylcyclohexyl]-1H-benzimidazol-5-yl)acetate,
(±) methyl 3-(1-[-3,3-dimethylcyclohexyl]-2-{[4-(trifluoromethoxy)phenyl]amino}-1H-benzimidazol-5-yl)propanoate,
methyl 3-(1-[(1S)-3,3-dimethylcyclohexyl]-2-{[4-(trifluoromethoxy)phenyl]amino}-1H-benzimidazol-5-yl)propanoate,
methyl 3-(1-[(1R)-3,3-dimethylcyclohexyl]-2-{[4-(trifluoromethoxy)phenyl]amino}-1H-benzimidazol-5-yl)propanoate,
(±) methyl 3-(1-[-3,3-dimethylcyclohexyl]-2-{[4-(propan-2-yloxy)phenyl]amino}-1H-benzimidazol-5-yl)propanoate,
methyl 3-(1-[(1 S)-3,3-dimethylcyclohexyl]-2-{[4-(propan-2-yloxy)phenyl]amino}-1H-benzimidazol-5-yl)propanoate,
methyl 3-(1-[(1R)-3,3-dimethylcyclohexyl]-2-{[4-(propan-2-yloxy)phenyl]amino}-1H-benzimidazol-5-yl)propanoate,
(±) methyl 3-(1-[-3,3-dimethylcyclohexyl]-2-{[4-(propan-2-yl)phenyl]amino}-1H-benzimidazol-5-yl)propanoate,
methyl 3-(1-[(1S)-3,3-dimethylcyclohexyl]-2-{[4-(propan-2-yl)phenyl]amino}-1H-benzimidazol-5-yl)propanoate,
methyl 3-(1-[(1R)-3,3-dimethylcyclohexyl]-2-{[4-(propan-2-yl)phenyl]amino}-1H-benzimidazol-5-yl)propanoate,
methyl 3-[2-{[4-(propan-2-yloxy)phenyl]amino}-1-(3,3,5,5-tetramethylcyclohexyl)-1H-benzimidazol-5-yl]propanoate,
methyl 3-[2-{[4-(propan-2-yl)phenyl]amino}-1-(3,3,5,5-tetramethylcyclohexyl)-1H-benzimidazol-5-yl]propanoate,
(±) methyl (6-methoxy-2-{[4-(propan-2-yl)phenyl]amino}-1-[(cis)-3,3,5-trimethylcyclohexyl]-1H-benzimidazol-5-yl)acetate,
methyl (6-methoxy-2-{[4-(propan-2-yl)phenyl]amino}-1-[(1S,5S)-3,3,5-trimethylcyclohexyl]-1H-benzimidazol-5-yl)acetate,
methyl (6-methoxy-2-{[4-(propan-2-yl)phenyl]amino}-1-[(1R,5R)-3,3,5-trimethylcyclohexyl]-1H-benzimidazol-5-yl)acetate,
(±) methyl (6-methoxy-2-{[4-(propan-2-yloxy)phenyl]amino}-1-[(cis)-3,3,5-trimethylcyclohexyl]-1H-benzimidazol-5-yl)acetate,
methyl (6-methoxy-2-{[4-(propan-2-yloxy)phenyl]amino}-1-[(1S,5S)-3,3,5-trimethylcyclohexyl]-1H-benzimidazol-5-yl)acetate,
methyl (6-methoxy-2-{[4-(propan-2-yloxy)phenyl]amino}-1-[(1R,5R)-3,3,5-trimethylcyclohexyl]-1H-benzimidazol-5-yl)acetate,
(±) methyl (6-methoxy-2-{[4-(trifluoromethoxy)phenyl]amino}-1-[(cis)-3,3,5-trimethylcyclohexyl]-1H-benzimidazol-5-yl)acetate,
methyl (6-methoxy-2-{[4-(trifluoromethoxy)phenyl]amino}-1-[(1S,5S)-3,3,5-trimethylcyclohexyl]-1H-benzimidazol-5-yl)acetate,
methyl (6-methoxy-2-{[4-(trifluoromethoxy)phenyl]amino}-1-[(1R,5R)-3,3,5-trimethylcyclohexyl]-1H-benzimidazol-5-yl)acetate,
methyl [6-fluoro-2-{[4-(propan-2-yl)phenyl]amino}-1-(3,3,5,5-tetramethylcyclohexyl)-1H-benzimidazol-5-yl]acetate,
methyl [6-fluoro-2-{[4-(propan-2-yloxy)phenyl]amino}-1-(3,3,5,5-tetramethylcyclohexyl)-1H-benzimidazol-5-yl]acetate,
methyl [6-fluoro-1-(3,3,5,5-tetramethylcyclohexyl)-2-{[4-(trifluoromethoxy)phenyl]amino}-1H-benzimidazol-5-yl]acetate,
(±) methyl (6-fluoro-2-{[4-(propan-2-yl)phenyl]amino}-1-[(cis)-3,3,5-trimethylcyclohexyl]-1H-benzimidazol-5-yl)acetate,
methyl (6-fluoro-2-{[4-(propan-2-yl)phenyl]amino}-1-[(1S,5S)-3,3,5-trimethylcyclohexyl]-1H-benzimidazol-5-yl)acetate,
methyl (6-fluoro-2-{[4-(propan-2-yl)phenyl]amino}-1-[(1R,5R)-3,3,5-trimethylcyclohexyl]-1H-benzimidazol-5-yl)acetate,
(±) methyl (6-fluoro-2-{[4-(propan-2-yloxy)phenyl]amino}-1-[(cis)-3,3,5-trimethylcyclohexyl]-1H-benzimidazol-5-yl)acetate,
methyl (6-fluoro-2-{[4-(propan-2-yloxy)phenyl]amino}-1-[(1S,5S)-3,3,5-trimethylcyclohexyl]-1H-benzimidazol-5-yl)acetate,
methyl (6-fluoro-2-{[4-(propan-2-yloxy)phenyl]amino}-1-[(1R,5R)-3,3,5-trimethylcyclohexyl]-1H-benzimidazol-5-yl)acetate,
(±) methyl (6-fluoro-2-{[4-(trifluoromethoxy)phenyl]amino}-1-[(cis)-3,3,5-trimethylcyclohexyl]-1H-benzimidazol-5-yl)acetate,
methyl (6-fluoro-2-{[4-(trifluoromethoxy)phenyl]amino}-1-[(1S,5S)-3,3,5-trimethylcyclohexyl]-1H-benzimidazol-5-yl)acetate,
methyl (6-fluoro-2-{[4-(trifluoromethoxy)phenyl]amino}-1-[(1R,5R)-3,3,5-trimethylcyclohexyl]-1H-benzimidazol-5-yl)acetate,
methyl [6-methoxy-2-{[4-(propan-2-yl)phenyl]amino}-1-(3,3,5,5-tetramethylcyclohexyl)-1H-benzimidazol-5-yl]acetate,
methyl [6-methoxy-2-{[4-(propan-2-yloxy)phenyl]amino}-1-(3,3,5,5-tetramethylcyclohexyl)-1H-benzimidazol-5-yl]acetate,
methyl [6-methoxy-1-(3,3,5,5-tetramethylcyclohexyl)-2-{[4-(trifluoromethoxy)phenyl]amino}-1H-benzimidazol-5-yl]acetate,
(±) (2-{[4-(trifluoromethoxy)phenyl]amino}-1-[(cis)-3,3,5-trimethylcyclohexyl]-1H-benzimidazol-5-yl)acetic acid,
(2-{[4-(trifluoromethoxy)phenyl]amino}-1-[(1S,5S)-3,3,5-trimethylcyclohexyl]-1H-benzimidazol-5-yl)acetic acid,
(2-{[4-(trifluoromethoxy)phenyl]amino}-1-[(1R,5R)-3,3,5-trimethylcyclohexyl]-1H-benzimidazol-5-yl)acetic acid,
(±) (2-{[4-(propan-2-yloxy)phenyl]amino}-1-[(cis)-3,3,5-trimethylcyclohexyl]-1H-benzimidazol-5-yl)acetic acid,
(2-{[4-(propan-2-yloxy)phenyl]amino}-1-[(1S,5S)-3,3,5-trimethylcyclohexyl]-1H-benzimidazol-5-yl)acetic acid,
(2-{[4-(propan-2-yloxy)phenyl]amino}-1-[(1R,5R)-3,3,5-trimethylcyclohexyl]-1H-benzimidazol-5-yl)acetic acid,
[1-(3,3,5,5-tetramethylcyclohexyl)-2-{[4-(trifluoromethoxy)phenyl]amino}-1H-benzimidazol-5-yl]acetic acid,
[2-{[4-(propan-2-yloxy)phenyl]amino}-1-(3,3,5,5-tetramethylcyclohexyl)-1H-benzimidazol-5-yl]acetic acid,
2-{[4-(propan-2-yl)phenyl]amino}-1-(3,3,5,5-tetramethylcyclohexyl)-1H-benzimidazol-5-yl]acetic acid,
(±) 3-(1-[3,3-dimethylcyclohexyl]-2-{[4-(trifluoromethoxy)phenyl]amino}-1H-benzimidazol-5-yl)propanoic acid,
3-(1-[(1S)-3,3-dimethylcyclohexyl]-2-{[4-(trifluoromethoxy)phenyl]amino}-1H-benzimidazol-5-yl)propanoic acid,
3-(1-[(1R)-3,3-dimethylcyclohexyl]-2-{[4-(trifluoromethoxy)phenyl]amino}-1H-benzimidazol-5-yl)propanoic acid,
(±) (2-{[4-(propan-2-yl)phenyl]amino}-1-[(cis)-3,3,5-trimethylcyclohexyl]-1H-benzimidazol-5-yl)acetic acid,
(2-{[4-(propan-2-yl)phenyl]amino}-1-[(1S,5S)-3,3,5-trimethylcyclohexyl]-1H-benzimidazol-5-yl)acetic acid,
(2-{[4-(propan-2-yl)phenyl]amino}-1-[(1R,5R)-3,3,5-trimethylcyclohexyl]-1H-benzimidazol-5-yl)acetic acid,
3-[2-{[4-(propan-2-yl)phenyl]amino}-1-(3,3,5,5-tetramethylcyclohexyl)-1H-benzimidazol-5-yl]propanoic acid,
(±) 3-(1-[3,3-dimethylcyclohexyl]-2-{[4-(propan-2-yl)phenyl]amino}-1H-benzimidazol-5-yl)propanoic acid,
3-(1-[(1S)-3,3-dimethylcyclohexyl]-2-{[4-(propan-2-yl)phenyl]amino}-1H-benzimidazol-5-yl)propanoic acid,
3-(1-[(1R)-3,3-dimethylcyclohexyl]-2-{[4-(propan-2-yl)phenyl]amino}-1H-benzimidazol-5-yl)propanoic acid,
(±) 3-(1-[3,3-dimethylcyclohexyl]-2-{[4-(propan-2-yloxy)phenyl]amino}-1H-benzimidazol-5-yl)propanoic acid,
3-(1-[(1S)-3,3-dimethylcyclohexyl]-2-{[4-(propan-2-yloxy)phenyl]amino}-1H-benzimidazol-5-yl)propanoic acid,
3-(1-[(1R)-3,3-dimethylcyclohexyl]-2-{[4-(propan-2-yloxy)phenyl]amino}-1H-benzimidazol-5-yl)propanoic acid,
3-[1-(3,3,5,5-tetramethylcyclohexyl)-2-{[4-(trifluoromethoxy)phenyl]amino}-1H-benzimidazol-5-yl]propanoic acid,
(±) (6-methyl-2-{[4-(trifluoromethoxy)phenyl]amino}-1-[(cis)-3,3,5-trimethylcyclohexyl]-1H-benzimidazol-5-yl)acetic acid,
(6-methyl-2-{[4-(trifluoromethoxy)phenyl]amino}-1-[(1S,5S)-3,3,5-trimethylcyclohexyl]-1H-benzimidazol-5-yl)acetic acid,
(6-methyl-2-{[4-(trifluoromethoxy)phenyl]amino}-1-[(1R,5R)-3,3,5-trimethylcyclohexyl]-1H-benzimidazol-5-yl)acetic acid,
(±) (6-methyl-2-{[4-(propan-2-yl)phenyl]amino}-1-[(cis)-3,3,5-trimethylcyclohexyl]-1H-benzimidazol-5-yl)acetic acid,
(6-methyl-2-{[4-(propan-2-yl)phenyl]amino}-1-[(1S,5S)-3,3,5-trimethylcyclohexyl]-1H-benzimidazol-5-yl)acetic acid,
(6-methyl-2-{[4-(propan-2-yl)phenyl]amino}-1-[(1R,5R)-3,3,5-trimethylcyclohexyl]-1H-benzimidazol-5-yl)acetic acid,
(±) (6-methyl-2-{[4-(propan-2-yloxy)phenyl]amino}-1-[(cis)-3,3,5-trimethylcyclohexyl]-1H-benzimidazol-5-yl)acetic acid,
(6-methyl-2-{[4-(propan-2-yloxy)phenyl]amino}-1-[(1S,5S)-3,3,5-trimethylcyclohexyl]-1H-benzimidazol-5-yl)acetic acid,
(6-methyl-2-{[4-(propan-2-yloxy)phenyl]amino}-1-[(1R,5R)-3,3,5-trimethylcyclohexyl]-1H-benzimidazol-5-yl)acetic acid,
[6-methyl-1-(3,3,5,5-tetramethylcyclohexyl)-2-{[4-(trifluoromethoxy)phenyl]amino}-1H-benzimidazol-5-yl]acetic acid,
[6-methyl-2-{[4-(propan-2-yl)phenyl]amino}-1-(3,3,5,5-tetramethylcyclohexyl)-1H-benzimidazol-5-yl]acetic acid,
[6-methyl-2-{[4-(propan-2-yloxy)phenyl]amino}-1-(3,3,5,5-tetramethylcyclohexyl)-1H-benzimidazol-5-yl]acetic acid,
[6-fluoro-2-{[4-(propan-2-yl)phenyl]amino}-1-(3,3,5,5-tetramethylcyclohexyl)-1H-benzimidazol-5-yl]acetic acid,
[6-fluoro-1-(3,3,5,5-tetramethylcyclohexyl)-2-{[4-(trifluoromethoxy)phenyl]amino}-1H-benzimidazol-5-yl]acetic acid,
[6-methoxy-2-{[4-(propan-2-yl)phenyl]amino}-1-(3,3,5,5-tetramethylcyclohexyl)-1H-benzimidazol-5-yl]acetic acid,
[6-methoxy-1-(3,3,5,5-tetramethylcyclohexyl)-2-{[4-(trifluoromethoxy)phenyl]amino}-1H-benzimidazol-5-yl]acetic acid,
(±) (6-methoxy-2-{[4-(trifluoromethoxy)phenyl]amino}-1-[(cis)-3,3,5-trimethylcyclohexyl]-1H-benzimidazol-5-yl)acetic acid,
(6-methoxy-2-{[4-(trifluoromethoxy)phenyl]amino}-1-[(1S,5S)-3,3,5-trimethylcyclohexyl]-1H-benzimidazol-5-yl)acetic acid,
(6-methoxy-2-{[4-(trifluoromethoxy)phenyl]amino}-1-[(1R,5R)-3,3,5-trimethylcyclohexyl]-1H-benzimidazol-5-yl)acetic acid,
(±) (6-methoxy-2-{[4-(propan-2-yl)phenyl]amino}-1-[(cis)-3,3,5-trimethylcyclohexyl]-1H-benzimidazol-5-yl)acetic acid,
(6-methoxy-2-{[4-(propan-2-yl)phenyl]amino}-1-[(1S,5S)-3,3,5-trimethylcyclohexyl]-1H-benzimidazol-5-yl)acetic acid,
(6-methoxy-2-{[4-(propan-2-yl)phenyl]amino}-1-[(1R,5R)-3,3,5-trimethylcyclohexyl]-1H-benzimidazol-5-yl)acetic acid,
(±) (6-methoxy-2-{[4-(propan-2-yloxy)phenyl]amino}-1-[(cis)-3,3,5-trimethylcyclohexyl]-1H-benzimidazol-5-yl)acetic acid,
(6-methoxy-2-{[4-(propan-2-yloxy)phenyl]amino}-1-[(1S,5S)-3,3,5-trimethylcyclohexyl]-1H-benzimidazol-5-yl)acetic acid,
(6-methoxy-2-{[4-(propan-2-yloxy)phenyl]amino}-1-[(1R,5R)-3,3,5-trimethylcyclohexyl]-1H-benzimidazol-5-yl)acetic acid,
(±) (6-fluoro-2-{[4-(propan-2-yl)phenyl]amino}-1-[(cis)-3,3,5-trimethylcyclohexyl]-1H-benzimidazol-5-yl)acetic acid,
(6-fluoro-2-{[4-(propan-2-yl)phenyl]amino}-1-[(1S,5S)-3,3,5-trimethylcyclohexyl]-1H-benzimidazol-5-yl)acetic acid,
(6-fluoro-2-{[4-(propan-2-yl)phenyl]amino}-1-[(1R,5R)-3,3,5-trimethylcyclohexyl]-1H-benzimidazol-5-yl)acetic acid,
(±) (6-fluoro-2-{[4-(propan-2-yloxy)phenyl]amino}-1-[(cis)-3,3,5-trimethylcyclohexyl]-1H-benzimidazol-5-yl)acetic acid,
(6-fluoro-2-{[4-(propan-2-yloxy)phenyl]amino}-1-[(1S,5S)-3,3,5-trimethylcyclohexyl]-1H-benzimidazol-5-yl)acetic acid,
(6-fluoro-2-{[4-(propan-2-yloxy)phenyl]amino}-1-[(1R,5R)-3,3,5-trimethylcyclohexyl]-1H-benzimidazol-5-yl)acetic acid,
(±) (6-fluoro-2-{[4-(trifluoromethoxy)phenyl]amino}-1-[(cis)-3,3,5-trimethylcyclohexyl]-1H-benzimidazol-5-yl)acetic acid,
(6-fluoro-2-{[4-(trifluoromethoxy)phenyl]amino}-1-[(1S,5S)-3,3,5-trimethylcyclohexyl]-1H-benzimidazol-5-yl)acetic acid,
(6-fluoro-2-{[4-(trifluoromethoxy)phenyl]amino}-1-[(1R,5R)-3,3,5-trimethylcyclohexyl]-1H-benzimidazol-5-yl)acetic acid,
methyl 3-[4-fluoro-1-(3,3,5,5-tetramethylcyclohexyl)-2-{[4-(trifluoromethoxy)phenyl]amino}-1H-benzimidazol-5-yl]propanoate,
methyl 3-{4-fluoro-2-[(4-isopropoxyphenyl)amino]-1-(3,3,5,5-tetramethylcyclohexyl)-1H-benzimidazol-5-yl}propanoate,
3-[4-fluoro-1-(3,3,5,5-tetramethylcyclohexyl)-2-{[4-(trifluoromethoxy)phenyl]amino}-1H-benzimidazol-5-yl]propanoic acid,
3-{4-fluoro-2-[(4-isopropoxyphenyl)amino]-1-(3,3,5,5-tetramethylcyclohexyl)-1H-benzimidazol-5-yl}propanoic acid,
(±) N,N-dimethyl-2-[(2-{[4-trifluoromethoxy)phenyl]amino}-1-[(cis)-3,3,5-trimethylcyclohexyl]-1H-benzimidazol-5-yl)oxy]acetamide,
N,N-dimethyl-2-[(2-{[4-trifluoromethoxy)phenyl]amino}-1-[(1S,5S)-3,3,5-trimethylcyclohexyl]-1H-benzimidazol-5-yl)oxy]acetamide,
N,N-dimethyl-2-[(2-{[4-trifluoromethoxy)phenyl]amino}-1-[(1R,5R)-3,3,5-trimethylcyclohexyl]-1H-benzimidazol-5-yl)oxy]acetamide,
(±) N-cyclopropyl-N-methyl-2-[(2-{[4-trifluoromethoxy)phenyl]amino}-1-[(cis)-3,3,5-trimethylcyclohexyl]-1H-benzimidazol-5-yl)oxy]acetamide,
N-cyclopropyl-N-methyl-2-[(2-{[4-trifluoromethoxy)phenyl]amino}-1-[(1S,5S)-3,3,5-trimethylcyclohexyl]-1H-benzimidazol-5-yl)oxy]acetamide, and
N-cyclopropyl-N-methyl-2-[(2-{[4-trifluoromethoxy)phenyl]amino}-1-[(1R,5R)-3,3,5-trimethylcyclohexyl]-1H-benzimidazol-5-yl)oxy]acetamide,

or a stereoisomer, a tautomer, an N-oxide, a hydrate, a solvate, or a salt thereof, or a mixture of same.

In accordance with a preferred embodiment, component A is selected from the group of:

(±) 3-(2-{[4-(trifluoromethoxy)phenyl]amino}-1-[(cis)-3,3,5-trimethylcyclohexyl]-1H-benzimidazol-5-yl)propanoic acid, or a stereoisomer, a tautomer, an N-oxide, a hydrate, a solvate, or a salt thereof, particularly a pharmaceutically acceptable salt thereof,

3-(2-{[4-(trifluoromethoxy)phenyl]amino}-1-[(1R,5R)-3,3,5-trimethylcyclohexyl]-1H-benzimidazol-5-yl)propanoic acid, or a tautomer, an N-oxide, a hydrate, a solvate, or a salt thereof, particularly a pharmaceutically acceptable salt thereof,

3-(2-{[4-(trifluoromethoxy)phenyl]amino}-1-[(1S,5S)-3,3,5-trimethylcyclohexyl]-1H-benzimidazol-5-yl)propanoic acid, or a tautomer, an N-oxide, a hydrate, a solvate, or a salt thereof, particularly a pharmaceutically acceptable salt thereof,

3-(2-{[4-(trifluoromethoxy)phenyl]amino}-1-[(cis)-3,3,5-trimethylcyclohexyl]-1H-benzimidazol-5-yl)propanoic acid enantiomer A, or a stereoisomer, a tautomer, an N-oxide, a hydrate, a solvate, or a salt thereof, particularly a pharmaceutically acceptable salt thereof,

3-(2-{[4-(trifluoromethoxy)phenyl]amino}-1-[(cis)-3,3,5-trimethylcyclohexyl]-1H-benzimidazol-5-yl)propanoic acid enantiomer B, or a stereoisomer, a tautomer, an N-oxide, a hydrate, a solvate, or a salt thereof, particularly a pharmaceutically acceptable salt thereof, and

(2E)-but-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4), preferably the crystalline form of (2E)-but-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4), which is characterized by maxima in the X-ray diffractogram at the following 2 theta angles: 5.4, 6.8, 10.2, 10.3, 10.8, 11.1, 16.8, 21.6.

In a more preferred embodiment, said component A is 3-(2-{[4-(trifluoromethoxy)phenyl]amino}-1-[(1R,5R)-3,3,5-trimethylcyclohexyl]-1H-benzimidazol-5-yl)propanoic acid, or a pharmaceutically acceptable salt thereof.

In a more preferred embodiment, said component A is (2E)-but-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4) (Compound A2).

Compounds of formula (1) as described and defined herein can be prepared according to methods known to a skilled person or according to the methods disclosed in the publications disclosed herein which are incorporated by reference in their entirety.

The mIDH1 inhibitors mentioned in the prior art as well as in the lists above have been disclosed for the treatment or prophylaxis of different diseases, especially cancer.

The specific compounds of the lists as disclosed above are preferred as being component A of the combination, most preferred are the compounds used in the experimental section.

A combination of the present invention comprising Compound A1 as mentioned above and a HMA is a preferred embodiment of the invention.

A combination of the present invention comprising Compound A2 as mentioned above and a HMA is a preferred embodiment of the invention.

Another embodiment of the present invention covers a combination comprising the Compound A1 or a pharmaceutically acceptable salt thereof as mentioned above and a HMA or a pharmaceutically acceptable salt thereof.

Another embodiment of the present invention covers a combination comprising the Compound A2 or a pharmaceutically acceptable salt thereof as mentioned above and a HMA or a pharmaceutically acceptable salt thereof.

Another embodiment of the present invention covers a combination comprising the Compound A1 or Compound A2, or a pharmaceutically acceptable salt thereof, and a HMA selected from the group consisting of:

decitabine, and

azacitidine,

or a stereoisomer, a tautomer, an N-oxide, a hydrate, a solvate, or a pharmaceutically acceptable salt thereof.

Another embodiment of the present invention covers a combination comprising the Compound A1 or Compound A2, or a pharmaceutically acceptable salt thereof, and a HMA selected from the group consisting of:

decitabine, and

azacitidine,

or a pharmaceutically acceptable salt thereof.

It is to be understood that the present invention relates also to any combination of the embodiments of component A described above.

Component A may be administered by the oral, intravenous, topical, local installations, intraperitoneal or nasal route.

Preferably Component A is administered intravenously, intraperitoneally or orally.

Compound A1 is administered preferably orally. Compound A2 is administered preferably orally.

Component B of the Combination

Component Bis a HMA, or a stereoisomer, a tautomer, an N-oxide, a hydrate, a solvate, or a pharmaceutically acceptable salt thereof.

Component B includes, but is not limited to, 5-aza-2′-deoxycytidine (decitabine), 5-azacytidine (azacitidine), 5,6-dihydro-5-azacytidine, and zebularine, or a stereoisomer, a tautomer, an N-oxide, a hydrate, a solvate, or a pharmaceutically acceptable salt thereof.

In accordance with a preferred embodiment, component Bis a HMA selected from the group consisting of:

decitabine, and

azacitidine,

or a stereoisomer, a tautomer, an N-oxide, a hydrate, a solvate, or a pharmaceutically acceptable salt thereof.

In accordance with a more preferred embodiment, component Bis a HMA selected from the group consisting of:

decitabine, and

azacitidine,

or a pharmaceutically acceptable salt thereof.

HMA according to the present invention are commercially available and/or can be prepared according to methods readily available to a skilled person. For example the following references, which are incorporated herein by reference, describe methods to prepare HMA and crystal forms and/or salts thereof:
decitabine, US20060014949, US 20060069060,
azacitidine, US20040186065, DE1140941, US20060069060, WO2009016617, for example.

Component B preferably is administered by the more appropriate route within the knowledge of the skilled person.

Component B may be administered by the oral, intravenous, topical, local installations, intraperitoneal or nasal route.

According to certain embodiments of the present invention Component Bis decitabine, or a stereoisomer, a tautomer, an N-oxide, a hydrate, a solvate, or a pharmaceutically acceptable salt thereof.

Decitabine (5-aza-2′-deoxycytidine, trade name Dacogen) is a hypomethylating agent approved in the US and Europe for the treatment of patients with myelodysplastic syndromes (MDS) including previously treated and untreated, de novo and secondary MDS of all French-American-British subtypes (refractory anemia, refractory anemia with ringed sideroblasts, refractory anemia with excess blasts, refractory anemia with excess blasts in transformation, and chronic myelomonocytic leukemia) and intermediate-1, intermediate-2, and high-risk International Prognostic Scoring System groups and/or acute myeloid leukaemia (AML). It hypomethylates DNA by inhibiting DNA methyltransferase.

Typically, in the first treatment cycle the recommended Dacogen dose is 15 mg/m2 administered by continuous intravenous infusion over 3 hours repeated every 8 hours for 3 days. Patients may be premedicated with standard anti-emetic therapy. In subsequent treatment cycles the above cycle is repeated every 6 weeks. It is recommended that patients be treated for a minimum of 4 cycles; however, a complete or partial response may take longer than 4 cycles. Treatment may be continued as long as the patient continues to benefit.

Alternatively, in a treatment cycle, Dacogen is administered at a dose of 20 mg/m2 body surface area by intravenous infusion over 1 hour repeated daily for 5 consecutive days (i.e., a total of 5 doses per treatment cycle). The total daily dose must not exceed 20 mg/m2 and the total dose per treatment cycle must not exceed 100 mg/m2. If a dose is missed, treatment should be resumed as soon as possible. The cycle should be repeated every 4 weeks depending on the patient's clinical response and observed toxicity. It is recommended that patients be treated for a minimum of 4 cycles; however, a complete or partial remission may take longer than 4 cycles to be obtained. Treatment may be continued as long as the patient shows response, continues to benefit or exhibits stable disease, i.e., in the absence of overt progression. If after 4 cycles, the patient's haematological values (e.g., platelet counts or absolute neutrophil count), have not returned to pre-treatment levels or if disease progression occurs (peripheral blast counts are increasing or bone marrow blast counts are worsening), the patient may be considered to be a non-responder and alternative therapeutic options to Dacogen should be considered. Dacogen (decitabine) for Injection is supplied as a sterile, lyophilized white to almost white powder, in a single-dose vial, packaged in cartons of 1 vial. Each vial contains 50 mg of decitabine.

The dosing and/or dosing regimen of decitabine may be adjusted according to patients's response, adverse events and or co-treatment with other drugs by the skilled person using methods readily available to him/her.

According to certain embodiments of the present invention Component Bis azacitidine, or a stereoisomer, a tautomer, an N-oxide, a hydrate, a solvate, or a pharmaceutically acceptable salt thereof.

Azacitidine (5-azacytidine; trade name Vidaza) is a hypomethylating agent approved in the US and Europe for the treatment of FAB myelodysplastic syndrome (MDS) subtypes: Refractory anemia (RA) or refractory anemia with ringed sideroblasts (RARS) (if accompanied by neutropenia or thrombocytopenia or requiring transfusions), refractory anemia with excess blasts (RAEB), refractory anemia with excess blasts in transformation (RAEB-T), and chronic myelomonocytic leukemia (CMMoL) and/or for the treatment of patients who are not eligible for haematopoietic stem cell transplantation (HSCT) with:
intermediate-2 and high-risk myelodysplastic syndromes (MDS) according to the International Prognostic Scoring System (IPSS);
chronic myelomonocytic leukaemia (CMML) with 10-29% marrow blasts without myeloproliferative disorder;
acute myeloid leukaemia (AML) with 20-30% blasts and multi-lineage dysplasia, according to World Health Organization (WHO) classification.

Typically, the recommended starting dose for the first treatment cycle, for all patients regardless of baseline hematology values, is VIDAZA 75 mg/m2 daily for 7 days to be administered by subcutaneous (SC) injection or intravenous (IV) infusion. Premedicate for nausea and vomiting. Repeat cycles every 4 weeks. After 2 cycles, may increase dose to 100 mg/m2 if no beneficial effect is seen and no toxicity other than nausea and vomiting has occurred. Patients should be treated for a minimum of 4 to 6 cycles. Complete or partial response may require additional treatment cycles.

Continue treatment as long as the patient continues to benefit. Patients should be monitored for hematologic response and renal toxicities, with dosage delay or reduction as appropriate. Vidaza is available as a lyophilized powder in 100 mg single-use vials. The dosing and/or dosing regimen of rucaparib may be adjusted according to patients's response, adverse events and or co-treatment with other drugs by the skilled person using methods readily available to him/her.

In accordance with an embodiment, the present invention relates to a combination of any component A mentioned herein with any component B mentioned herein, optionally with any component C mentioned herein.

Further, the present invention relates to:

a kit comprising:
a combination of:
component A: one or more mIDH1 inhibitors, or a stereoisomer, a tautomer, an N-oxide, a hydrate, a solvate, or a pharmaceutically acceptable salt thereof;
component B: a HMA, or a stereoisomer, a tautomer, an N-oxide, a hydrate, a solvate, or a pharmaceutically acceptable salt thereof; and, optionally,
component C: one or more further pharmaceutical agents;

in which optionally either or both of said components A and Bin any of the above-mentioned combinations are in the form of a pharmaceutical formulation which is ready for use to be administered simultaneously, concurrently, separately or sequentially.

The term “component C” being at least one pharmaceutical agent includes the effective compound itself as well as its pharmaceutically acceptable salts, solvates, hydrates or stereoisomers as well as any composition or pharmaceutical formulation comprising such effective compound or its pharmaceutically acceptable salts, solvates, hydrates or stereoisomers. A list of such readily available agents is being provided further below.

The components may be administered together or independently of one another by the oral, intravenous, topical, local installations, intraperitoneal or nasal route.

Component C being administered as the case may be.

Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

Components of this invention can also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the component in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It can be desirable or necessary to introduce a component of the present invention to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin); oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide); tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylenestearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 g/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/L sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 g of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 g of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Commercial Utility

Component A

The compounds of formula (1) or pharmaceutically acceptable salts, solvates, hydrates or stereoisomers thereof according to the combination as referred to above are components A. The compounds according to the combination have valuable pharmaceutical properties, which make them commercially utilizable. In particular, they inhibit mIDH1 and are expected to be commercially applicable in the therapy of diseases (e.g. cancer).

Component B

Due to the mechanism as discussed in the introductory section component Bis especially suitable to have effects on tumor diseases. In particular, they are DNA hypomethylating agents (HMA) and are commercially applicable in the therapy of the diseases indicated herein or in other indications known to a skilled person.

Combination

The combinations of the present invention thus can be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

One embodiment relates to the use of a combination according to the invention for the preparation of a medicament for the treatment or prophylaxis of a cancer, particularly MDS or AML, and/or metastases thereof. Particularly preferred is the use of a combination according to the invention for the preparation of a medicament for the treatment or prophylaxis of the specific subtypes of MDS or subtypes of AML described herein, and/or metastases thereof.

In one embodiment the invention relates to a method of treatment or prophylaxis of a cancer, particularly MDS or AML, and/or metastases thereof, in a subject, comprising administering to said subject a therapeutically effective amount of a combination according to the present invention. Preferred types of cancer are those subtypes of MDS or subtypes of AML described herein, and/or metastases thereof.

One preferred embodiment is the use of the combinations of the invention for the treatment of the disorders tested in the experimental section.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Combinations of the present invention might be utilized to inhibit, block, reduce, decrease, etc., cell proliferation and/or cell division, and/or produce apoptosis.

This invention includes a method comprising administering to a mammal in need thereof, including a human, an amount of a component A, or a stereoisomer, a tautomer, an N-oxide, a hydrate, a solvate, or a pharmaceutically acceptable salt thereof, and an amount of component B of this invention, or a stereoisomer, a tautomer, an N-oxide, a hydrate, a solvate, or a pharmaceutically acceptable salt thereof; which is effective to treat the disorder, such as MDS or AML, and/or metastases thereof, particularly the subtypes of MS or subtypes of AML described herein, and/or metastases thereof.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Dose and Administration

Component A

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 30 mg/kg body weight per day. The total amount of the active ingredients per dose will generally range from about 1 mg to about 500 mg per dose, and preferably from about 20 mg to about 200 mg per dose. Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Component B

The HMA can be administered as described above or, alternatively using an alternative dose and dose regimen as can be readily determined by a skilled person using known techniques.

The specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a HMA of the present invention or a stereoisomer, a tautomer, an N-oxide, a hydrate, a solvate, or a pharmaceutically acceptable salt thereof, or a composition thereof can be ascertained by those skilled in the art using conventional treatment tests.

Suitable dose(s), administration regime(s) and administration route(s) for HMAs include those defined in the Patient Information Leaflet or in Clinical Practice Guidelines in Oncology. Alternatively, suitable dose(s), administration regime(s) and administration route(s) for HMA may be readily determined by standard techniques known to the skilled person.

The dose(s), administration regime(s) and administration route(s) may have to be adapted according to, inter alia, the indication, the indication stage, the patient age and/or the patient gender, among other factors. Such adaptations can be readily determined by standard techniques known to the skilled person.

For both the mIDH1 inhibitors and the HMAs of the present invention the administered dosage and/or administration regime may be modified, independently of each other or simultaneously, depending on any superior or unexpected results which may be obtained as routinely determined with this invention.

The HMA can be administered to a patient orally, topically, parenterally, rectally, by inhalation, and by injection. Administration by injection includes intravenous, intramuscular, subcutaneous, and parenterally as well as by infusion techniques. The agents can be administered by any of the conventional routes of administration for these compounds. The preferred route of administration is typically the same route of administration used for the agent when used alone.

For administering the mIDH1 inhibitor and the HMA, by any of the routes of administration herein discussed, the mIDH1 inhibitor can be administered simultaneously with the HMA. This can be performed by administering a single formulation which contains both the mIDH1 inhibitor and the HMA or by administering the mIDH1 inhibitor and HMA in independent formulations at the same time (concomittantly) to a patient.

Alternatively, the mIDH1 inhibitor can be administered in tandem with the HMA. The mIDH1 inhibitor can be administered prior to the HMA inhibitor. Also, the HMA inhibitor can be administered first followed by adminstration of the mIDH1 inihibitor. The choice of sequence administration of the mIDH1 inihibitor relative to the HMA may vary for different agents, and can be readily determined and, when needed, modified or adapted by the skilled person using techniques readily available in order, for example, to improve the therapeutic effect of the combination. Also, the HMA can be administered using any regimen which is conventionally used for these agents.

In an embodiment, administration of component B starts simultaneously or on the same day (i.e. in paralell) with component A, or starts on different days (i.e. sequential).

In another regimen of administration, the mIDH1 inihibitor and the HMA can be administered once or more times per day on the day(s) of administration.

Any of the routes and regimens of administration may be modified depending on any superior or unexpected results which may be obtained as routinely determined with this invention.

The combinations of component A and component B of this invention can be administered as the sole pharmaceutical agent or in combination with one or more further pharmaceutical agents C where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

Optional anti-hyper-proliferative agents which can be added as component C to the combination of components A and B of the present invention include but are not limited to compounds listed on the cancer chemotherapy drug regimens in the 14^{th} Edition of the Merck Index, (2006), which is hereby incorporated by reference.

Other anti-hyper-proliferative agents suitable for use as component C with the combination of components A and B of the present invention include but are not limited to those compounds acknowledged to be used in the treatment of neoplastic diseases in Goodman and Gilman's The Pharmacological Basis of Therapeutics (12th Edition), editor Brunton et al., publ. by McGraw-Hill, pages 1667-1769, (2011), which is hereby incorporated by reference.

Generally, the use of cytotoxic and/or cytostatic agents as component C in combination with a combination of components A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemo-therapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(8) provide a longer time for tumor progression, and/or
(9) yield efficacy and tolerability results at least as good as those of the agents used alone, compared to known instances where other cancer agent combinations produce antagonistic effects.

### Experimental Section

In the present study, we assessed the combination of the DNA hypomethylating agent azacitidine with mutated IDH1 inhibitor Compound A1 in a preclinical PDX model of IDH1 mutated AML.

1. Preparation of IDH1 Inhibitors and HMA

The schemes and procedures described in the art as cited in the present application disclose general synthetic routes and specific procedures to synthesize the mIDH1 inhibitor compounds which are preferred components A of the present combination.

Specifically, compound A1, 3-(2-{[4-(trifluoromethoxy)phenyl]amino}-1-[(1R,5R)-3,3,5-trimethylcyclohexyl]-1H-benzimidazol-5-yl)propanoic acid can be prepared according to the methods described in WO2015/121210:

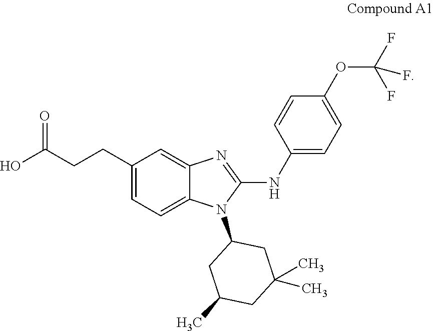 [SMILES: C[C@H]1C[C@@H](n2c(Nc3ccc(OC(F)(F)F)cc3)nc3cc(CCC(=O)O)ccc32)CC(C)(C)C1]

Compound A2, the adduct (2E)-but-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4) can be prepared according to the methods described in WO2017/016992.

Similarly, the HMA agents which are preferred component B of the present combination are described in the art and/or are available commercially, particularly azacitidine and decitabine.

2. Methods: We tested the activity of Compound A1 alone or in combination with escalating doses of azacitidine in IDH1 mutated and IDH1 wildtype AML patient cells in vitro evaluating the effects on colony formation, apoptosis, cell cycle, differentiation and global gene expression changes.

Clonogenic Progenitor Assay:

Colony-forming cell (CFC) units were assayed in methylcellulose (Methocult H4100; StemCell Technologies Inc.) supplemented with 10 ng/mL IL3, 10 ng/mL GM-CSF, 50 ng/mL SCF, 50 ng/mL FLT3-ligand and 3 U/mL EPO (PeproTech). 100 nm of Compound A1 was added in combination with increasing concentrations of Azacytidine to methylcellulose containing 10exp5 human mononuclear cells and were plated in duplicate. Colonies were evaluated microscopically 10 to 14 days after plating by standard criteria. Graphs was plotted as proportion of colonies relative to vehicle treated cells.

Cell Cycle Analysis:

For cell cycle analysis 10exp5 to 10exp6 IDH1mut or IDH1wt human mononuclear from AML patients were treated with either 100 nM Compound A1 and 100 nM Azacytidine alone or in combination. 10 μM of BrdU was added after 64 hours and at 72 hours after treatment the cells were harvested, fixed, permeabilized, and stained with an anti-BrdU antibody according to the manufacturers protocol (BD Pharmingen Cat no. 559619). Cell cycle phases were determined according to standard procedures where BrdU positive cells are in the S phase of the cell cycle.

Transplantation and Treatment of Mice:

Leukemic cells from an AML patient with mutated DH1, NPM1, FLT3-TKD and NRAS were xenografted in immunocompromised mice. One million patient-derived AML cells collected from bone marrow and spleen of leukemic mice were injected intravenously in the tail vein of sublethally (3 Gy) irradiated NSG mice. Treatment was initiated 28 days after transplantation, when engraftment of human cells (hCD45+ cells) in peripheral blood had been confirmed. The proportion of leukemic cells in peripheral blood of mice was measured with a human-specific CD45 antibody by tail vein bleeds and FACS analysis. The control groups were treated with either vehicle, Compound A1 150 mg/kg once daily p.o. continuously, or azacitidine 1 mg/kg once daily s.c. days 1-5, repeated once after 28 days. The test groups were treated with Compound A1 and azacitidine in the doses mentioned above either starting both drugs on day 1 (parallel group) or starting azacitidine on day 1 but Compound A1 on day 6 (sequential group). The treatment was stopped after 84 days (FIG. 5).

Limiting Dilution Transplantation

For limiting dilution transplantation, primary mice with high chimerism (70-80%) were treated for 4 weeks with either vehicle, azacitidine, BAY 1436032, or the sequential or simultaneous combination of BAY 1436032 and azacitidine. After 4 weeks of treatment bone marrow cells were obtained from primary mice and injected intravenously at doses of 2,000,000, 200,000, 20,000, 2,000, 200 or 20 cells (n=3 mice/dose) into irradiated secondary NSG recipients. Eight weeks after transplantation, the presence of transplanted human cells in peripheral blood was assessed by flow cytometry. Engraftment was determined positive when more than 0.1% hCD45+ cells were detected. The frequency of stem cells was calculated by applying Poisson statistics to the proportion of negative recipients at different dilutions using the ELDA software Gene Expression Profiling

For gene expression profiling RNA was extracted using the RNeasy Plus mini kit (Qiagen) from hCD45+ cells that were sorted from bone marrow of PDX mice 4 weeks after treatment with vehicle, azacitidine (1 mg/kg, s.c., days 1 to 5), BAY1436032 (150 mg/kg, p.o., q.d., 4 weeks) or the simultaneous combination of BAY 1436032 and azacitidine. Control of quality and integrity of total RNA, biotin labeling, hybridization on Affymetrix GeneChip HG-U133 2.0, staining, image analysis and analysis of microarray data was performed.

3. Results: Compound A1 reduced colony formation specifically in human IDH1 mutated AML cells, while IDH1 wildtype cells were not affected (IC50 100 nM). Combination of Compound A1 (100 nM) with azacitidine (100 nM) further reduced colony formation by 50% in IDH1 mutated AML cells, while the IC50 for IDH1 wildtype AML cells was not reached even at 1000 nM azacitidine in the presence of 100 nM Compound A1 (FIG. 1). For in vivo experiments human AML cells from an IDH1 mutated AML patient were transplanted into sublethally irradiated NSG mice and treatment was started four weeks after transplantation (FIG. 5). Leukemic cells in peripheral blood constantly increased in vehicle and azacitidine treated mice, while leukemic cells declined from week 4 until the end of treatment at week 12 in mice treated with Compound A1 alone as well as in the parallel and sequential combination groups treated with Compound A1 and azacitidine. However, in week 20 (8 weeks after stopping the Compound A1 treatment) leukemia relapsed in the group with Compound A1 alone as well as in the group with the sequential combination. Interestingly, the frequency of leukemic cells remained low in peripheral blood in mice treated in parallel with the combination of Compound A1 and azacitidine even at 24 weeks after starting the treatment (FIG. 2A, Band for individual animals see FIG. 3B) and the combination treatment caused a survival benefit of the animals compared to the animals reated in monotherapy (FIG. 3A). To obtain mechanistic insights into the efficacy of the combination treatment we treated human IDH1 mutant and IDH1 wildtype AML cells in vitro with either 100 nm Compound A1 or 100 nm azacitidine alone or in combination (in parallel). Vehicle treatment served as control. There were no significant differences in percentage of apoptotic cells between Compound A1 or azacitidine treatment as single agents or in combination. However, the proportion of cells in S phase of the cell cycle was synergistically decreased by the combination treatment compared with either monotherapy or vehicle (FIG. 4). Additionally the combination treatment with BAY1436032 and azacitidine strongly depletes leukemia stem cells in vivo through inhibition of MAP-kinase signaling and activation of myeloid differentiation

4. Conclusion: Our study provides the first evidence of synergistic activity of an IDH1 inhibitor with hypomethylating agents and strongly argues for simultaneous application (or on the same day) of the IDH1 inhibitor Compound A1 or Compound A2 with azacitidine or decitabine in future clinical trials. Clinical development is ongoing with phase 1 studies using Compound A1 in IDH1 mutant solid tumors and AML.

### BRIEF DESCRIPTION OF THE FIGURES

FIG. 1: Colony formation assay with leukemic cells from a patient with AML with mutated (mut) IDH1 and from a patient with IDH1 wildtype (wt) treated with Compound A1 (aka BAY) and azacitidine (aka AZA) alone or in combination.

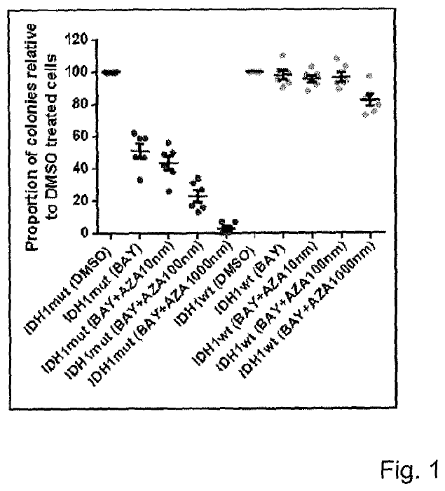

FIG. 2A: Development of human IDH1 mutated AML in peripheral blood of NSG mice treated with Compound A1 (aka BAY) and azacitidine (aka AZA) alone or in combination (PRL, in parallel; SEQ, sequential, i.e. first azacitidine days 1-5, then BAY from day 6 onwards). Leukemia is significantly delayed in PDX AML mice treated with the combination of Compound A1 and azacitidine in parallel compared to sequential treatment or monotherapy.

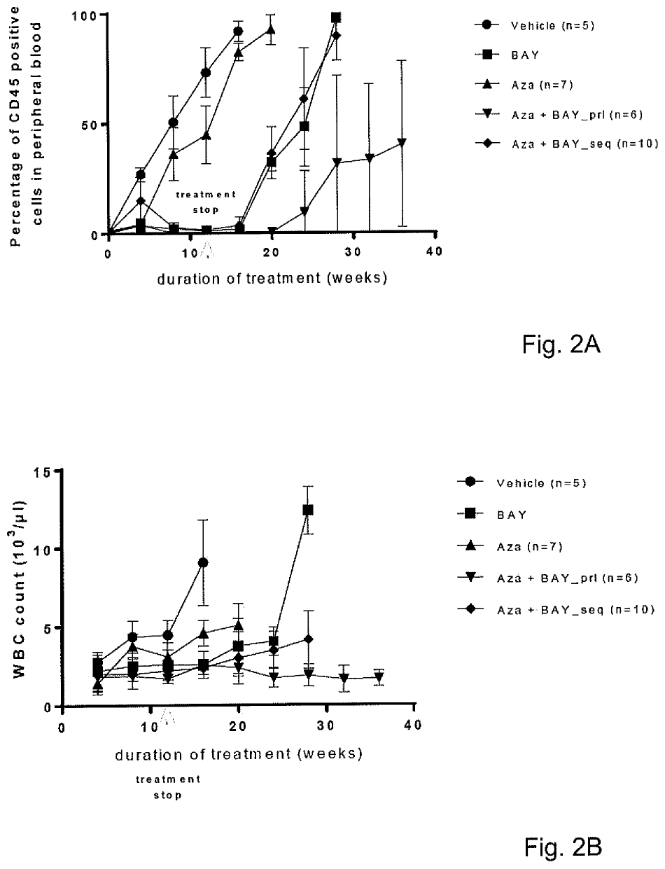

FIG. 2B: Development of white blood count (WBC) in peripheral blood of NSG mice treated with Compound A1 (aka BAY) and azacitidine (aka AZA) alone or in combination (PRL, in parallel; SEQ, sequential, i.e. first azacitidine days 1-5, then BAY from day 6 onwards).

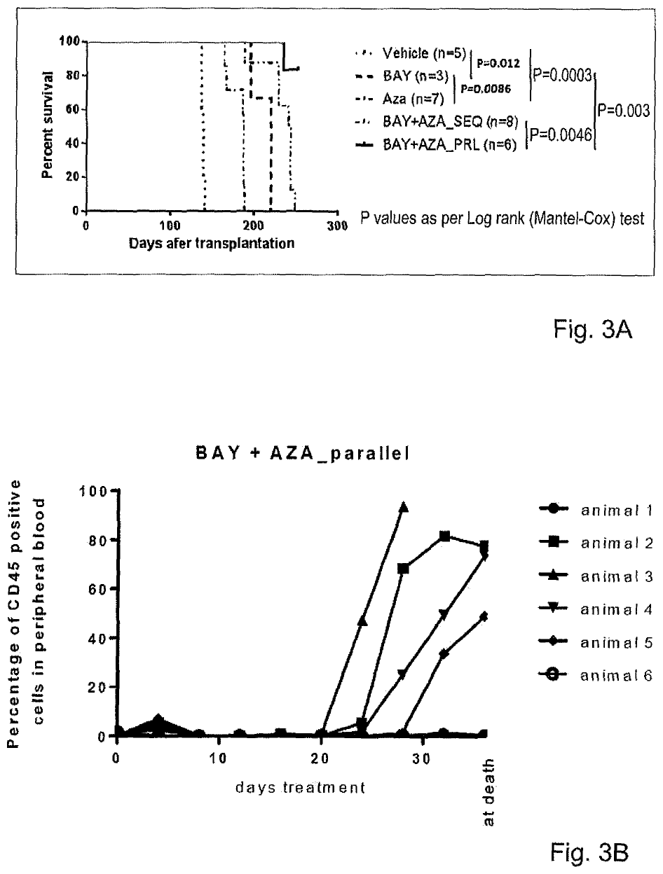

FIG. 3A: Survival of NSG mice with human IDH1 mutated AML in peripheral blood treated with Compound A1 (aka BAY) and azacitidine (aka AZA) alone or in combination (PRL, in parallel; SEQ, sequential, i.e. first azacitidine days 1-5, then BAY from day 6 onwards). Combined treatment of Compound A1 with azacitidine in parallel significantly increased the survival of mice transplanted with IDH1mut AML cells.

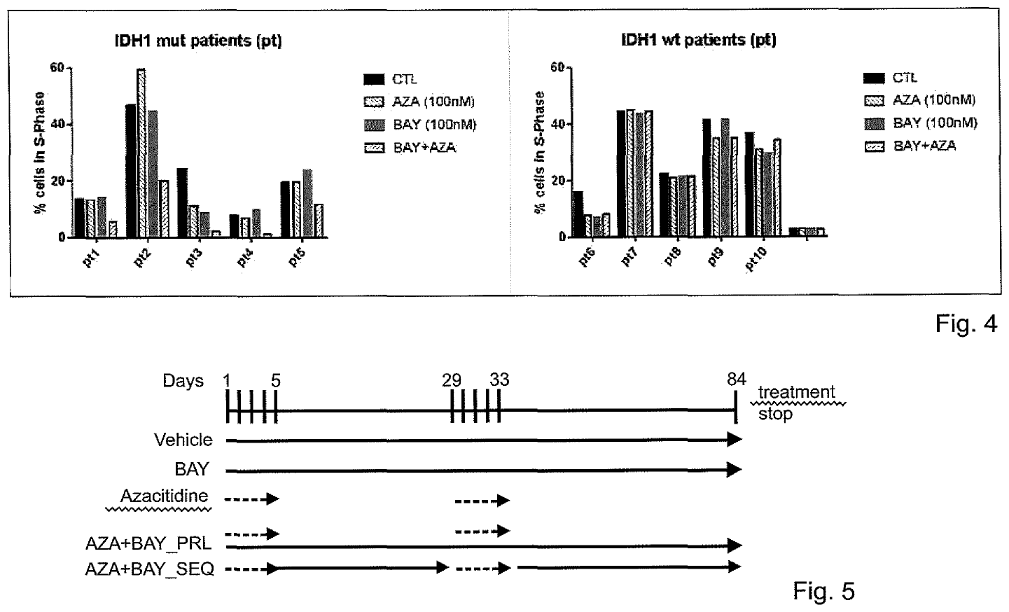

FIG. 3B: Development of human CD45+ human AML cells in peripheral blood of individual NSG mice treated with Compound A1 (aka BAY) and azacitidine (aka AZA) in combination in parallel. With 2/6 mice have less than 3% leukemic cells in peripheral blood at death.

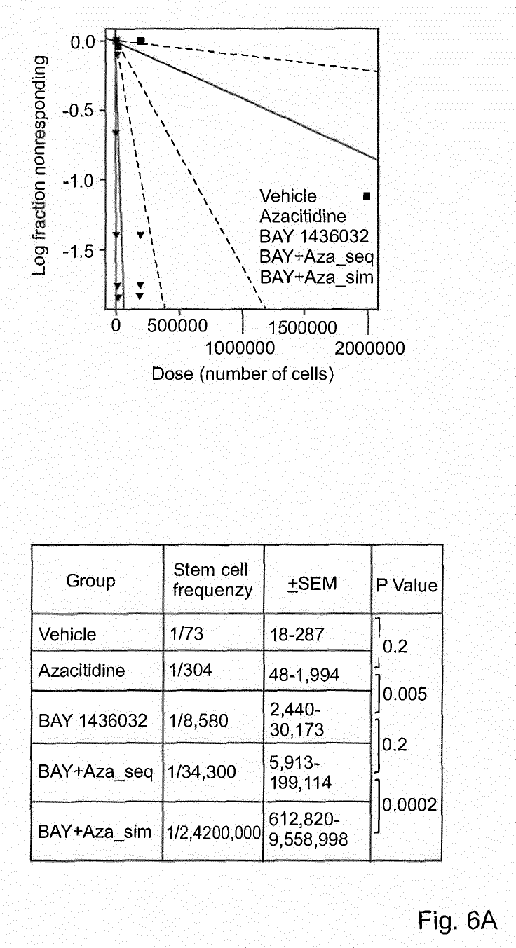

FIG. 4: Cell cycle assay with leukemic cells from a patient with AML with mutated IDH1 and from a patient with IDH1 wildtype treated with Compound A1 (aka BAY) and azacitidine (aka AZA) alone or in combination.

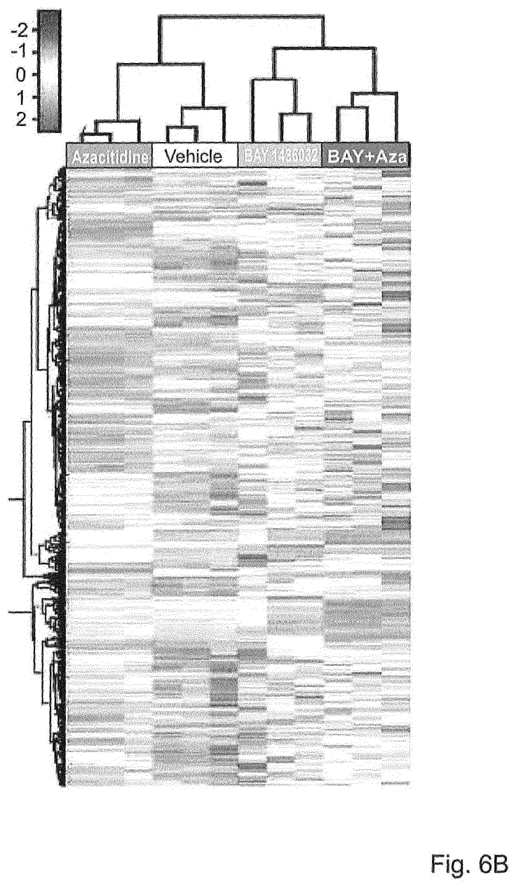

FIG. 5: Study design of Transplantation and treatment of mice study. Treatment was started 28 days after transplantation, i.e. Day 1 occurs 29 days after transplantation. BAY=Compound A1, AZA=azacitidine, PRL=in parallel; SEQ=sequential.

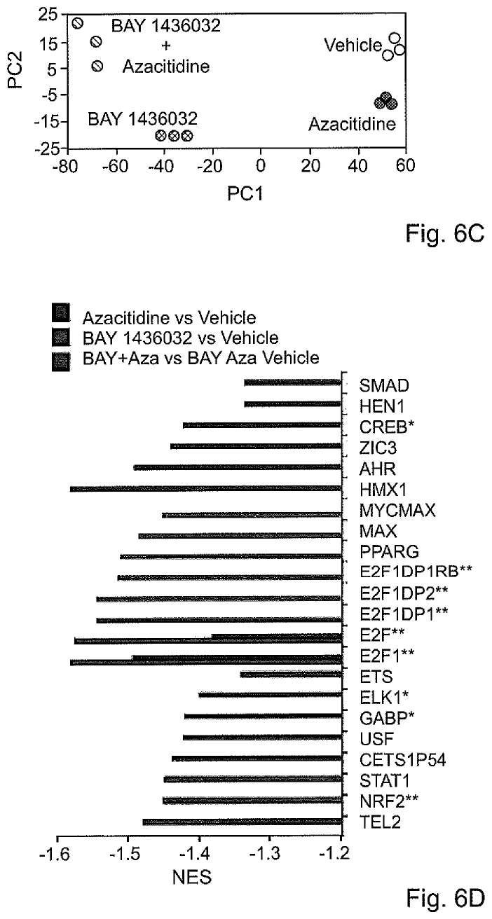

FIG. 6: Combination treatment with BAY 1436032 and azacitidine strongly depletes leukemia stem cells in vivo through inhibition of MAP-kinase signaling and activation of myeloid differentiation. (A) Limiting dilution transplantation of bone marrow cells from IDH1 mutant PDX mice treated with vehicle, azacitidine (1 mg/kg, s.c., days 1 to 5), BAY1436032 (150 mg/kg, p.o., q.d., 4 weeks) or the sequential or simultaneous combination of BAY1436032 and azacitidine with the same doses as in the single agent treated mice. 2,000,000, 200,000, 20,000, 2,000, 200 or 20 human AML cells per mouse were transplanted into 3 recipient mice per cell dose. LSC frequencies are shown (mean±SEM, n=3). Mice with hCD45+ cells in peripheral blood after 8 weeks (>0.1%) were scored positive. The estimated stem cell frequency after 4 weeks of treatment is given in the table. (B) Unsupervised hierarchical clustering using euclidean distance of cells from bone marrow of IDH1 mutant PDX mice treated with vehicle, azacitidine (1 mg/kg, s.c., days 1 to 5), BAY1436032 (150 mg/kg, p.o., q.d., 4 weeks) or the simultaneous combination of BAY 1436032 and azacitidine. Cells were harvested from bone marrow at 4 weeks after treatment and sorted for hCD45+ cells. Gene expression profiling using RNA was performed on Affymetrix Human HG_U133 Plus 2.0 microarrays (n=3 per group). (C) Principal component analysis of all treatment groups using the top 4000 differentially expressed genes. (D) Gene set enrichment analysis (MSigDB version 6.0) showing the most enriched transcription factor target gene sets from the indicated treatment comparisons. NES, normalized enrichment score. * gene sets involved in MAP Kinase signaling; ** gene sets involved in RB/E2F signaling (E) Heatmap from gene expression levels of MAP kinase signaling genes, RB/E2F signaling genes and myeloid differentiation genes from the bone marrow of IDH1 mutant PDX mice treated with vehicle, azacitidine (1 mg/kg, s.c., days 1 to 5), BAY1436032 (150 mg/kg, p.o., q.d., 4 weeks) or the simultaneous combination of BAY1436032 and azacitidine. Gene expression was determined by quantitative RT-PCR relative to the housekeeping gene ABL and was normalized to gene expression in vehicle-treated cells (mean±SEM, n=3). (F) Representative western blots of in vitro cultured HT1080, a fibrosarcoma cell line with an endogenous heterozygous IDH1R132C mutation treated with vehicle, azacitidine, BAY 1436032 or the simultaneous combination of BAY1436032 and azacitidine using antibodies against the indicated signaling proteins.

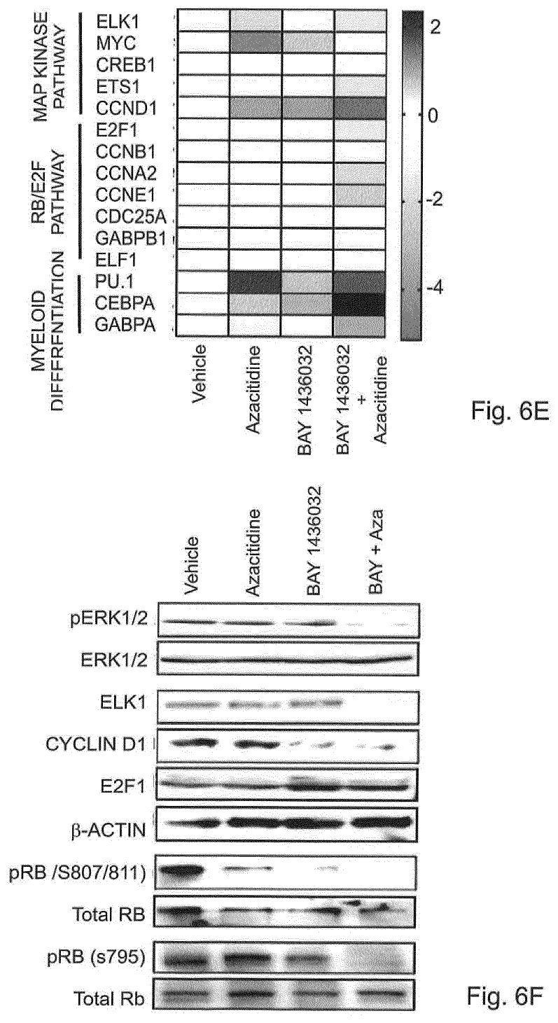

1. A combination of at least two components, comprising:
(a) a component A selected from 3-(2-{[4-(trifluoromethoxy)phenyl]amino}-1-[(1R,5R)-3,3,5-trimethylcyclohexyl]-1H-benzimidazol-5-yl)propanoic acid and (2E)-But-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4), or a stereoisomer, a tautomer, an N-oxide, a hydrate, a solvate, or a pharmaceutically acceptable salt thereof, and
(b) a component B being a hypomethylating agent (HMA), wherein the HMA is azacitidine or decitabine, or a stereoisomer, a tautomer, an N-oxide, a hydrate, a solvate, or a pharmaceutically acceptable salt thereof.

2. The combination of claim 1, wherein the component A is 3-(2-{[4-(trifluoromethoxy)phenyl]amino}-1-[(1R,5R)-3,3,5-trimethylcyclohexyl]-1H-benzimidazol-5-yl)propanoic acid, or a pharmaceutically acceptable salt thereof.

3. The combination of claim 1, wherein the component A is (2E)-But-2-enedioic acid-3-(2-{[4-(trifluoromethoxy)phenyl]amino}-1-[(1R,5R)-3,3,5-trimethylcyclohexyl]-1H-benzimidazol-5-yl)propanoic acid (1:4).

4. The combination of claim 1, wherein the component B is Azacitidine, or a stereoisomer, a tautomer, an N-oxide, a hydrate, a solvate, or a pharmaceutically acceptable salt thereof.

5. The combination of claim 1, wherein the component B is Decitabine, or a stereoisomer, a tautomer, an N-oxide, a hydrate, a solvate, or a pharmaceutically acceptable salt thereof.

6. The combination of claim 1, for the treatment of acute myeloid leukemia (AML) and/or metastases thereof, wherein the AML is isocitratdehydrogenase 1 (IDH1) mutated AML and/or metastases thereof.

7. The combination of claim 1 for the preparation of a medicament for the treatment or prophylaxis of AML and/or metastases thereof, particularly IDH1 mutated AML and/or metastases thereof.

8. A method of treatment or prophylaxis of a AML and/or metastases thereof in a subject, comprising administering to the subject a therapeutically effective amount of the combination of claim 1; wherein the AML is IDH1 mutated AML and/or metastases thereof.

9. The combination of claim 6, wherein the treatment with the component A and the component B starts simultaneously or on the same day or starts on different days.

10. A pharmaceutical composition, comprising the combination of claim 1 together with a pharmaceutically acceptable ingredient.

11. The combination of claim 7 or the method according to claim 8, wherein the treatment with the component A and the component B starts simultaneously or on the same day or starts on different days.

12. The method of claim 8, wherein the treatment with the component A and the component B starts simultaneously or on the same day or starts on different days.
